(19) United States
(12) Patent Application Publication
(10) Pub. No.: US 2024/0299448 A1
(43) Pub. Date: Sep. 12, 2024

(54) METHODS FOR PREVENTING AND TREATING CHRONIC PAIN

(71) Applicants: Luda DIATCHENKO, Montreal (CA); Jeffrey MOGIL, Montreal West (CA); Lucas Vasconcelos LIMA, Montreal (CA); Marc PARISIEN, Montreal (CA); Massimo ALLEGRI, Bergamo (IT); Nader GHASEMLOU, Kingston (CA); Mohamad KARAKY, Montreal (CA)

(72) Inventors: Luda DIATCHENKO, Montreal (CA); Jeffrey MOGIL, Montreal West (CA); Lucas Vasconcelos LIMA, Montreal (CA); Marc PARISIEN, Montreal (CA); Massimo ALLEGRI, Bergamo (IT); Nader GHASEMLOU, Kingston (CA); Mohamad KARAKY, Montreal (CA)

(21) Appl. No.: 18/181,865

(22) Filed: Mar. 10, 2023

(51) Int. Cl. A61K 35/15; A61K 31/197; A61K 31/485; A61K 31/56; A61K 38/18; A61K 38/19; A61K 38/20; A61K 38/21; A61P 25/04

(52) U.S. Cl. CPC A61K 35/15; A61K 31/197; A61K 31/485; A61K 31/56; A61K 38/1866; A61K 38/191; A61K 38/2006; A61K 38/204; A61K 38/2053; A61K 38/208; A61K 38/21; A61P 25/04

(57) ABSTRACT

The present invention relates to a method of preventing or treating chronic pain comprising administering to a subject, in an amount/number effective to elicit, sustain or potentiate an inflammatory process, a pro-inflammatory compound, myeloid leukocyte cells or a substance that increases the number or activity of myeloid leukocyte cells.

### TECHNICAL FIELD

The present invention pertains generally to the prevention and treatment of pain. More specifically, it pertains for example to methods for preventing and/or treating chronic pain, to a corresponding kit and to a method of producing an animal model of chronic pain.

### BACKGROUND OF THE INVENTION

Chronic pain inflicts huge societal costs, both in terms of management, loss of work productivity, and effects on quality of life (Gereau et al., 2014). Chronic low back pain (LBP) is the most frequently reported chronic pain condition (Schopflocher et al., 2011). LBP is a major problem worldwide: point, 1-month, and 1-year prevalence are 18%, 31%, and 38%, respectively (Hoy et al., 2012). LBP ranks highest of all chronic conditions in terms of years lived with disability, with its prevalence and burden increasing with age (G. D. a. I. I. a. P. Collaborators, 2018). Current treatments for LBP often target the immune system and include non-steroidal anti-inflammatory drugs (NSAIDs), acetaminophen, and corticosteroids, although all of these drug classes are minimally effective at best (Chou et al., 2017). Despite advances in the understanding of social, psychological, and genetic factors, as well as brain processes associated with chronic LBP (Vlaeyen et al., 2018), in the prior there has been only very little understanding of the molecular mechanisms underlying the acute-to-chronic pain transition, preventing the development of more efficacious analgesic strategies.

Previous human genetic association studies and transcriptomic analysis of chronic LBP have been performed using candidate gene and genome-wide approaches, and they have provided evidence for the involvement of a variety of genes in many biological pathways (Freidin et al., 2019; Ramesh et al., 2018; Starkweather et al., 2016; Dorsey et al., 2019; Freidin et al., 2021). Increasing evidence suggests that the pathophysiology of chronic pain involves a complex interplay between the nervous and immune systems; that is, chronic pain is a neuroinflammatory disorder mediated by neuronal and non-neuronal cells alike (Ji et al., 2016). Circulating immune cells such as neutrophils, monocytes, and T cells are recruited to sites of tissue damage and/or inflammation, and often also infiltrate the peripheral and central nervous systems (Ji et al., 2016; Kavelaars and Heijnen, 2021). Activation of these cells results in the expression of various inflammatory mediators, including cytokines/chemokines, lipids, and proteases, that act both directly on peripheral sensory or central second order neurons, and indirectly on other immune or local cells to regulate pain. Microglia and astrocytes in the CNS act in a similar fashion, contributing to central sensitization and pain (Scholz and Woolf, 2007; Ji et al., 2013; Grace et al., 2014; Grace et al., 2021). The presence of these activated immune cells and glia, peripherally or centrally, is thought to contribute to the transition from acute to chronic pain (Ji et al., 2018; Chapman and Vierck, 2017; Mifflin and Kerr, 2014).

In view of the above, there is an unmet medical need in the management of chronic pain. In particular, the prevention and/or treatment of chronic pain is still unsatisfactory. It is thus the object of the present invention to provide novel means for preventing or treating chronic pain. Preferably, these means have at least one of the following advantages over current treatments for chronic pain, such as treatments for LBP: They are preferably more efficacious, safer, more widely applicable and/or reduce the burden on the healthcare system.

### SUMMARY OF THE INVENTION

In one embodiment, the present invention pertains to a method of preventing or treating chronic pain in a subject in need thereof, comprising: administering to said subject one or more of:
a. a pro-inflammatory compound in an amount effective to elicit, sustain or potentiate an inflammatory process in said subject,
b. myeloid leukocyte cells in a number effective to elicit, sustain or potentiate an inflammatory process in said subject, and
c. a substance that increases the number or activity of myeloid leukocyte cells in an amount effective to elicit, sustain or potentiate an inflammatory process in said subject,

thereby preventing or treating said chronic pain.

Said myeloid leukocyte cells are preferably selected from the group consisting of granulocytes, monocytes and macrophages. Preferably, said granulocytes are neutrophils. Thus, said myeloid leukocyte cells are more preferably neutrophils, monocytes and macrophages, and most preferably neutrophils.

Preferably, one or more of said pro-inflammatory compound, said myeloid leukocyte cells and said substance are administered during a phase of acute pain or during a phase of chronic pain.

Said administered pro-inflammatory compound is preferably selected from neutrophil-derived factors.

A preferred pro-inflammatory compound is selected from the group consisting of an alarmin, a cytokine, a growth factor, a prostaglandin receptor agonist and combinations thereof. Preferably, the alarmin is selected from the group consisting of S100A8 and S100A9. A preferred cytokine is selected from the group consisting of IL-1α, IL-1β, IL-6, IL-8, TNF-α, IFN-γ, IL-12 and IL-18. Preferably, the growth factor is selected from the group consisting of EREG, VEGF and GM-CSF. A preferred prostaglandin receptor agonist is selected from the group consisting of a PTGIR agonist and a PTGER4 agonist.

In a preferred method according to this embodiment of the present invention, said administered myeloid leukocyte cells, or said myeloid leukocyte cells whose number or activity is increased, degranulate and/or release a pro-inflammatory compound. Preferably, the pro-inflammatory compound released by said myeloid leukocyte cells is an alarmin, in particular selected from the group consisting of S100A8 and S100A9.

Preferably, said substance that increases the number or activity of myeloid leukocyte cells (in particular neutrophils) is a chelator of Ca²⁺ ions.

Preferably, in the method according to this embodiment of the present invention, said inflammatory process elicited, sustained or potentiated is transient.

In a preferred method according to this embodiment of the present invention, said chronic pain is selected from the group consisting of a neuropathic pain state, a myofascial pain state, an inflammatory pain state, chronic postoperative pain, low back pain and temporomandibular disorder.

In another embodiment, the present invention pertains to a method of preventing or treating chronic pain in a subject in need thereof, comprising: eliciting, sustaining, potentiating or preserving an inflammatory process in said subject, thereby preventing or treating said chronic pain.

Preferably, eliciting, sustaining or potentiating said inflammatory process comprises administering to said subject one or more of
an effective amount of a pro-inflammatory compound,
an effective number of myeloid leukocyte cells, and
an effective amount of a substance that increases the number or activity of myeloid leukocyte cells.

Preferably, said myeloid leukocyte cells are selected from the group consisting of granulocytes, monocytes and macrophages. Preferably, said granulocytes are neutrophils. Thus, said myeloid leukocyte cells are more preferably neutrophils, monocytes and macrophages, and most preferably neutrophils.

It is preferred that said inflammatory process is elicited, sustained, potentiated or preserved during a phase of acute pain or during a phase of chronic pain.

In a preferred method according to this embodiment of the invention, said inflammatory process elicited, sustained, potentiated or preserved is transient.

Said chronic pain is preferably selected from the group consisting of a neuropathic pain state, a myofascial pain state, an inflammatory pain state, chronic postoperative pain, low back pain and temporomandibular disorder.

In a further embodiment, the present invention relates to a method of treating acute pain and concomitantly preventing chronic pain in a subject suffering from acute pain, comprising: classifying said subject as a subject at risk of developing chronic pain due to an insufficient inflammatory process, and administering to said subject an analgesic in an amount effective to treat said acute pain, wherein said analgesic elicits substantially no anti-inflammatory effect at the amount administered, thereby treating said acute pain and concomitantly preventing said chronic pain.

Said analgesic is preferably an analgesic having substantially no anti-inflammatory properties. Preferred analgesics having substantially no, or no, anti-inflammatory properties are gabapentin, an opioid, lidocaine, acetaminophen (paracetamol) and an antidepressant.

Preferably, in the method according to this embodiment of the present invention, the analgesic is selected from the group consisting of gabapentin, an opioid, lidocaine, acetaminophen (paracetamol) and an antidepressant.

Preferably, classifying said subject involves isolating blood from said subject, determining the amount of one or more inflammation markers in the isolated blood, and, provided that the determined amount of one, several or all inflammation markers is lower than a respective pre-determined threshold, classifying said subject as a subject at risk of developing chronic pain due to an insufficient inflammatory process.

An example for such a pre-determined threshold is the level of C-Reactive Protein (CRP). CRP is preferably detected in blood or in plasma. Usually, a threshold of 2-3 mg/L is used to detect an inflammatory process. Thus, said pre-determined threshold is preferably 2-3 mg/L of CRP, such as 2 mg/L, 2.5 mg/L or 3 mg/L.

In the method according to this embodiment of the present invention, said acute pain is preferably selected from the group consisting of a neuropathic pain state, a myofascial pain state, an inflammatory pain state, postoperative pain, low back pain and temporomandibular disorder.

In yet another embodiment, the present invention pertains to a method of treating acute pain and concomitantly preventing chronic pain in a subject suffering from acute pain, comprising: administering to said subject a combination of (i) an analgesic in an amount effective to treat said acute pain and (ii) one or more of
a pro-inflammatory compound in an amount effective to elicit, sustain or potentiate an inflammatory process in said subject,
myeloid leukocyte cells in a number effective to elicit, sustain or potentiate an inflammatory process in said subject, and
a substance that increases the number or activity of myeloid leukocyte cells in an amount effective to elicit, sustain or potentiate an inflammatory process in said subject,
thereby treating said acute pain and concomitantly preventing said chronic pain.

Said myeloid leukocyte cells are preferably selected from the group consisting of granulocytes, monocytes and macrophages. Preferably, said granulocytes are neutrophils. Thus, said myeloid leukocyte cells are more preferably neutrophils, monocytes and macrophages, and most preferably neutrophils.

For example, in the method according to this embodiment of the present invention, said analgesic may be an analgesic having or not having anti-inflammatory properties. Preferably, said analgesic is selected from the group consisting of an NSAID, a corticosteroid, gabapentin, an opioid, lidocaine, acetaminophen (paracetamol) and an antidepressant. A preferred NSAID is selected from the group consisting of acetylsalicylic acid, flurbiprofen, ketoprofen, ketorolac, tolmetin, ibuprofen, naproxen, celecoxib, diclofenac, etodolac, indomethacin, mefenamic acid, meloxicam, piroxicam, sulindac, etoricoxib and lumiracoxib. Preferably, said corticosteroid is selected from the group consisting of betamethasone, cortisol (hydrocortisone), cortisone, dexamethasone, methylprednisolone, prednisolone, prednisone and triamcinolone. Preferably, said opioid is selected from the group consisting of alfentanil, buprenorphine, carfentanyl, codeine, diacetylmorphine (heroin), dihydrocodeine, dihydroetorphine, etorphine, fentanyl, hydrocodone, hydromorphone, levomethadone, meptazinol, methadone, morphine, nalbuphine, ohmefentanyl, oxycodone, pentazocine, pethidine, piritramide, remifentanil, sufentanil, tapentadol, tilidine and tramadol.

In the method according to this embodiment of the present invention, said acute pain is preferably selected from the group consisting of a neuropathic pain state, a myofascial pain state, an inflammatory pain state, postoperative pain, low back pain and temporomandibular disorder.

In a further embodiment, the present invention pertains to a method of treating acute pain and concomitantly preventing chronic pain in a subject suffering from acute pain, comprising: classifying said subject as a subject eligible for being administered an analgesic having anti-inflammatory properties and also eligible for being administered an analgesic having substantially no anti-inflammatory properties, and administering to said subject the analgesic having substantially no anti-inflammatory properties but no analgesic having anti-inflammatory properties, thereby treating said acute pain and preventing said chronic pain.

In an additional embodiment, the present invention relates to a method of classifying acute or chronic pain afflicting a subject, comprising:
isolating blood from said subject, determining the amount of one or more inflammation markers in the isolated blood, and, provided that the determined amount of one, several or all inflammation markers is lower than a respective pre-determined threshold, classifying said pain afflicting the subject as acute or chronic pain whose chronification is to be prevented or treated, respectively, and/or
isolating neutrophils from said subject, determining a first degree of activity of said isolated neutrophils, subsequently contacting said isolated neutrophils with a substance that increases the activity of neutrophils, determining a second degree of activity of said isolated neutrophils after being contacted with said substance, and, provided that the second degree of activity is higher than the first degree of activity and preferably that the second degree of activity is higher than a pre-determined threshold, classifying said pain afflicting the subject as acute or chronic pain whose chronification is to be prevented or treated, respectively.

As concerns the predefined thresholds, reference is made to the disclosure above.

Said substance that increases the activity of neutrophils is preferably selected from the group consisting of LPS, zymosan, PMA, TNF-α and IL-8. Said substance that increases the activity of neutrophils may be preferably selected from TLR ligands.

In the method according to this embodiment of the present invention, the first and/or second degree of activity is preferably determined by determining the amount of one or more inflammation markers.

Preferably, the inflammation markers whose amount is detected are selected from the group consisting of S100A8, S100A9, IL-1α, IL-1β, IL-6, TNF-α, IFN-γ, IL-12, IL-18, IL-8, EREG and GM-CSF.

The above option b may also be performed in the following alternative way:
isolating myeloid leukocyte cells from said subject, determining a first degree of activity of said isolated myeloid leukocyte cells, subsequently contacting said isolated myeloid leukocyte cells with a substance that increases the activity of myeloid leukocyte cells, determining a second degree of activity of said isolated myeloid leukocyte cells after being contacted with said substance, and, provided that the second degree of activity is higher than the first degree of activity and preferably that the second degree of activity is higher than a pre-determined threshold, classifying said pain afflicting the subject as acute or chronic pain whose chronification is to be prevented or treated, respectively,
in which case the above statements concerning the substance that increases the activity of neutrophils apply correspondingly.

In a further embodiment, the present invention relates to a method of preventing or treating chronic pain in a subject suffering from acute or chronic pain, comprising: classifying the pain afflicting said subject according to the method according to the preceding embodiment of the present invention, and, provided that the pain afflicting said subject has been classified as acute or chronic pain whose chronification is to be prevented or treated, respectively, administering to said subject
a pro-inflammatory compound in an amount effective to elicit, sustain or potentiate an inflammatory process in said subject,
myeloid leukocyte cells in a number effective to elicit, sustain or potentiate an inflammatory process in said subject or
a substance that increases the number or activity of myeloid leukocyte cells in an amount effective to elicit, sustain or potentiate an inflammatory process in said subject,
thereby preventing or treating said chronic pain.

Said myeloid leukocyte cells are preferably selected from the group consisting of granulocytes, monocytes and macrophages. Preferably, said granulocytes are neutrophils. Thus, said myeloid leukocyte cells are more preferably neutrophils, monocytes and macrophages, and most preferably neutrophils.

Said inflammatory process elicited, sustained or potentiated is preferably transient.

In another embodiment, the present invention relates to a method of producing an animal model of chronic pain, comprising:
providing an animal,
producing a pain state in the animal,
administering to the animal, or inducing in the animal the production of, one or more of an anti-inflammatory compound and a substance that decreases the number or activity of myeloid leukocyte cells, or treating the animal so as to decrease the number or activity of myeloid leukocyte cells,
thereby producing an animal model of chronic pain.

It is preferred that said myeloid leukocyte cells are selected from the group consisting of granulocytes, monocytes and macrophages. Preferably, said granulocytes are neutrophils. Thus, said myeloid leukocyte cells are more preferably neutrophils, monocytes and macrophages, and most preferably neutrophils.

Said administering, inducing or treating is preferably carried out from the time of producing the pain state. Additionally or independently, said administering, inducing or treating is preferably carried out for 2, 3, 4, 5, 6, 7, 8, 10, 12, 14, 16, 18 or 20 days or more than 20 days.

Preferably, said pain state is measurable by measuring mechanical allodynia. Mechanical allodynia is preferably measured with an esthesiometer. A preferred esthesiometer is a von Frey filament. Preferably, the up-down method (Dixon), also called the staircase method, is used.

Said pain state preferably persists for 40, 50, 60, 70, 80, 90, 100, 110 or 120 days or longer.

Producing the pain state in the animal preferably comprises
administering to said animal an immunostimulant in an amount effective to produce an inflammatory pain state,
applying a foreign body to a nerve of said animal so as to produce a neuropathic pain state, or
administering to said animal a neurotrophic factor in an amount effective to produce a myofascial pain state.

Preferably, the immunostimulant is complete Freund's adjuvant. In a preferred method, applying the foreign body to said nerve of said animal comprises applying one or more ligatures around the nerve so as to produce chronic constriction injury. A preferred neurotrophic factor is NGF.

In the method of producing an animal model of chronic pain according to the present invention, said animal preferably exhibits a reduced production of pro-inflammatory compounds. Preferably, the animal is a rodent. A preferred animal is a transgenic animal.

Said anti-inflammatory compound is preferably selected from the group consisting of an NSAID and a corticosteroid. Preferably, the substance that decreases the number or activity of myeloid leukocyte cells is an anti-Ly6G antibody. Said treating is preferably carried out by cryotherapy. Cryotherapy is preferably performed for 2, 3 or 4 days. The duration of each cryotherapy treatment is preferably from 15 to 90 min, more preferably from 30 to 60 min. Cryotherapy is preferably applied once, two times, three times or four times a day.

In a further embodiment, the present invention pertains to an animal produced by the method of producing an animal model of chronic pain according to the invention.

In yet another embodiment, the present invention relates to a method of modulating a pain state in an animal, comprising:
providing the animal produced by the method of producing an animal model of chronic pain according to the invention, wherein the animal has a pain state,
administering to the animal one or more of:
a pro-inflammatory compound in an amount effective to elicit, sustain or potentiate an inflammatory process in said animal,
myeloid leukocyte cells in a number effective to elicit, sustain or potentiate an inflammatory process in said animal, and
a substance that increases the number or activity of myeloid leukocyte cells in an amount effective to elicit, sustain or potentiate an inflammatory process in said animal,
thereby modulating said pain state.

Preferably, said myeloid leukocyte cells are selected from the group consisting of granulocytes, monocytes and macrophages. Said granulocytes are preferably neutrophils. Thus, said myeloid leukocyte cells are more preferably neutrophils, monocytes and macrophages, and most preferably neutrophils.

In a further embodiment, the present invention pertains to a kit comprising a combination of (i) an analgesic and (ii) one or more of a pro-inflammatory compound, myeloid leukocyte cells and a substance that increases the number or activity of myeloid leukocyte cells.

Preferably, the kit comprises an effective amount of said analgesic. It is also preferred that the kit comprises an effective amount of said pro-inflammatory compound, an effective number of said myeloid leukocyte cells or an effective amount of said substance that increases the number or activity of myeloid leukocyte cells.

### DESCRIPTION OF THE DRAWINGS

FIG. 1. Low back pain transcriptomics study design. (A) Study design. 98 patients reporting substantial back pain at enrolment (to) were assessed three months later at follow-up (t₁). At t₁, half of participants self-reported averaged daily pain scores of 4 and above, or persistent pain (P), whereas the other half—below 4, or resolved pain (R). Spontaneous resolution of pain is depicted in R (thick lines), whereas persistency in P (thin lines). (B) Pain score trajectories between visits. Trajectories marked by pain outcome: resolved pain in thick lines, whereas persistent pain in thin lines. Numbers indicate participant counts in each pain score slots. (C-F) Transcriptomics analysis contrasts enabled by the study design. A total of four contrasts have been made in which the transcriptomes. Large dots indicate the conditions that are compared. Study design pictograms are: (C) at t₀, between persistent and resolved pain outcomes, (D) at t₁, between persistent and resolved, (E) in those with persistent pain, between clinical visits t₁ and t₀. (F) in those with resolved pain, between t₁ and t₀.

FIG. 2. Epidemiological data related to the pain outcome at the second visit and sex. Values in parentheses are standard deviations. Abbreviations are: resolving pain group, R; persistent pain group, P; male, M; female, F; first visit, to; second visit, t₁; yes, y; no, n; body mass index, BMI; numeric rating scale, NRS; total score pain detect, TSPD; anti-inflammatory drug, AID; nonsteroidal AID, NSAID; corticosteroid AID, CSAID; total score pain detect, TSPD; not applicable, N/A. Among those taking medication, those taking medication in a prophylaxis fashion before the first visit, prophy.

FIGS. 3-6. Differential expression of genes in study design contrasts. Study design pictograms (left panels) are juxtaposed to volcano plots (middle and right panels). The pictogram highlights the two contrasted conditions (large dots). The volcano plot shows statistical significance (y axis) as a function of fold change (x axis); each dot is a gene. Genes that would end up outside of the plot are squeezed inside. Vertical grey line indicates null fold change. Genes reaching statistical significance at the FDR 10% level are found above the horizontal line at Y=1. Numbers in bold indicate counts of significantly differentially expressed genes that are down-regulated (lower left corner) or up-regulated (lower right corner). Volcano plots are shown uncorrected (middle column) and corrected (right column) for blood cell type fractions. FIG. 3, 4: Contrast between patients with persistent (P) and resolved (R) pain outcomes (FIG. 3) at t₀ or (FIG. 4) at t₁. FIG. 5, 6: Contrast between t₁ and t₀ in P patients (FIG. 5), or in R patients (FIG. 6).

FIGS. 7-11. Blood cell type fraction trajectories in time, in subjects with persistent or resolved pain. FIG. 7: Study design pictograms, showing contrasts in time in those with persistent pain (P, red, column I), and in those with resolved pain (R, green, column II). FIG. 8-11: Box-and-whisker plots (columns I and II) showing the distributions of percent cell type fraction at t₀ and at t₁. Cell type fraction estimates inferred by CIBERSORT from transcriptomics data. Statistical significance P-value obtained from logistic regression between the two time points shown on top; not significant (n.s.) when P>0.05. Volcano plots (column III) for R patients, showing genes highly expressed in the corresponding row's cell type (volcano dots and wide density lines) versus all other genes (narrow density lines). FIG. 8: Neutrophils. FIG. 9: CD8+ T cells. FIG. 10: Natural killer (NK) cells, resting. FIG. 11: Mast cells, resting.

FIG. 12. Functional differences between the resolved and persistent pain groups. Functional differences assessed with selected pathways in Gene Ontology's (GO) biological processes. Statistically significant pathways at the FDR 10% level are highlighted in non-grey colors; blue at t₀, (column I), red for group P (column III), and green for group R (column IV). Assessment performed between the groups with resolved (R) and persistent (P) pain, at the first visit (column I), and at the second visit (column II). Functional trajectories between t₀ and t₁ are also shown for the P group (column III), and for the R group (column IV). (A) Pathways under inflammatory response (GO:0006954). (B) Pathways under leukocyte activation (GO:0045321). (C) Pathways under leukocyte degranulation (GO:0043299).

FIGS. 13-16. Prolongation of neuropathic and inflammatory pain by early anti-inflammatory treatment. FIG. 13 left: Mechanical pain thresholds before and after chronic constriction injury (CCI) of the sciatic nerve in mice treated from day 0-6 with saline or dexamethasone (DEXA). Symbols represent mean t SEM hind paw withdrawal threshold (g). FIG. 13 middle: Percentage of maximum possible allodynia (% allodynia) on day 6 post-drug; see Methods for calculation details. Error bars represent SEM. FIG. 13 right: Days required to return to baseline thresholds; see Methods. Error bars represent SEM. Mice not returning to baseline by day 70 were assigned a value of 80. FIG. 14 left: Mechanical pain thresholds before and after hind paw injection of nerve growth factor (NGF) into the muscles of the low back in mice treated with saline or DEXA. Symbols as in FIG. 13 left. FIG. 14 middle: % allodynia on day 6 post-drug. FIG. 14 right: Days to return to baseline; mice not returning to baseline on day 50 were assigned a value of 60. FIG. 15 left: Mechanical pain thresholds before and after hind paw injection of complete Freund's adjuvant (CFA) in mice treated with saline or DEXA. Symbols as in FIG. 13 left. FIG. 15 middle % allodynia on day 6 post-drug. FIG. 15 right: Days to return to baseline; mice not returning to baseline on day 120 were assigned a value of 140. FIG. 16 left: Mechanical pain thresholds before and after CFA in mice treated with saline, diclofenac, gabapentin, or lidocaine. Symbols as in FIG. 13 left. FIG. 16 middle: % allodynia on day 4 post-drug. FIG. 16 right: Days to return to baseline; mice not returning to baseline on day 40 were assigned a value of 50. *P<0.05, **P<0.01, ***P<0.001 compared to corresponding saline group.

FIGS. 17-18. Involvement of neutrophils and neutrophil-released proteins S100A8 and S100A9 in pain resolution in the mouse. FIG. 17 left: Mechanical pain thresholds before and after CFA in mice treated with anti-Ly6G antibody or its isotype control (vehicle). Symbols represent mean±SEM hind paw withdrawal threshold (g). FIG. 17 middle: % allodynia on day 6 post-drug; see Methods for calculation details. FIG. 17 right: Days to return to baseline; see Methods. Mice not returning to baseline on day 120 were assigned a value of 140. FIG. 18 left: Mechanical pain thresholds before and after CFA in mice treated with DEXA (or saline), plus a hind paw injection of vehicle, isolated neutrophils, S100A8 or S100A9 on day 3 and 5 post-DEXA. Symbols as in FIG. 17 left. FIG. 18 right: % allodynia on day 6 post-DEXA. *P<0.05, **P<0.01, ***P<0.001 compared to corresponding vehicle/No DEXA group.

FIG. 19. Overlap of highly expressed genes in selected blood cell types. The overlap is quantified using a 4-way Venn diagram. Cell type specific gene expression from LM22 expression matrix of CIBERSORT. Figure drawn with the online tool interactivenn.net.

FIG. 20. Overlap between leading-edge genes in the leukocyte activation, leukocyte degranulation and inflammatory response pathways. The overlap is quantified using a 3-way Venn diagram. Figure drawn with the online tool www.interactivenn.net.

FIG. 21. Concerted pathway trajectories in time between the two pain groups. Each dot is a pathway. Pathway coordinates are in test statistic space, obtained from pathway analyses by fgsea. Pathway density increases towards the center. The slanted thick line was obtained from a linear regression of the data, whereas the slanted thin line from theoretical expectation of equal trajectories. Percent variance explained (Pearson's r²), slope (m), and P-value of regression are reported.

FIG. 22-23. Replication of the time evolution of genes and pathways, in subjects with persistent or resolved pain. FIG. 22 top: Study design pictograms, showing contrasts in time in those with persistent pain (P, thin lines), and in those with resolved pain (R, thick lines). FIG. 22 bottom: Differential gene expression in time. The differential gene expression is tracked in those with resolved pain (thick lines) and with persistent pain (thin lines) separately, with magnitude of the differential expression measured by the test statistic of the difference at gene-level. Upper panels show normalized histograms or density plots, whereas lower panels show cumulative fractions of the same data. Kolmogorov-Smirnov test P-value for difference between P and R groups indicated at the bottom right corners. Left: Data from the discovery cohort for low back pain (LBP). Right: Data from the replication cohort for temporomandibular disorders (TMD). FIG. 23: Differential pathway expression in time. fgsea's enrichment scores (ES) and corresponding enrichment P-values (pval) are shown for the discovery (LBP) and replication (TMD) cohorts. Meta-analysis (meta) of combined Z-scores (Z) and corresponding P-values (pval) are also shown. Far right column indicates if the pathway has been replicated. N.A. stands for not applicable.

FIG. 24. Contrasts of pathways between healthy subjects and those with pain. Contrast shown for selected pathways in the OPPERA TMD cohort; inflammatory response (GO:0006954), neutrophil activation (GO:0042119) and degranulation (GO:0043312). fgsea's enrichment score (ES) and associated P-value (pval) shown for each one of the four study design end points and with those with chronic pain (variable) against healthy controls (fixed).

FIG. 25. Prolongation of CCI-induced allodynia by diclofenac. Left: Mechanical pain thresholds before and after chronic constriction injury (CCI) of the sciatic nerve in mice treated from day 0-6 with saline or diclofenac (Diclo.). Symbols represent mean t SEM hind paw withdrawal threshold (g). Middle: Percentage of maximum possible allodynia (% allodynia) on day 6 post-drug; see Methods for calculation details. Error bars represent SEM. Right Days required to return to baseline thresholds; see Methods. Error bars represent SEM. Mice not returning to baseline by day 100 were assigned a value of 120. *P<0.05 compared to saline.

FIG. 26. No effect on CFA-induced mechanical allodynia of neutrophils and S100A8/A9 in the absence of dexamethasone. Left: Mechanical pain thresholds before and after injection of complete Freund's adjuvant (CFA) in mice treated on day 3 and day 5 with neutrophils, S100A8, or S100A9, but (in contrast to FIG. 5C) in the absence of dexamethasone (DEXA). Symbols represent mean±SEM hind paw withdrawal threshold (g). Right: Percentage of maximum possible allodynia (% allodynia) on day 6 post-drug; see Methods for calculation details. Error bars represent SEM. Neither the repeated measures ANOVA nor comparisons at day 6 (or any other time point) showed differences compared to vehicle.

FIG. 27. Paw edema after CFA Injection. After CFA injection, paw edema in the mice was measured as variation in dorso-palmar diameter (Δ in dorso-palmar diameter).

FIG. 28. Withdrawal thresholds of mice treated with cryotherapy after CFA Injection. After CFA injection, withdrawal thresholds of mice treated with cryotherapy were determined versus controls. Mice received cryotherapy over three days for either 30 min or 60 min. Top: cryotherapy once per day for 60 min, bottom: cryotherapy three times per day for 30 min each.

FIG. 29. A PTGIR agonist prevents prolongation of postoperative pain caused by dexamethasone. Mechanical pain thresholds before and after plantar incision in the ipsilateral hind paw comparing allodynia in four groups of CD-1 mice: a PBS control group, a DEXA+PBS group, a DEXA+PTGIR agonist group, and a group treated only with PTGIR agonist (4 mice per group). Symbols represent mean±SEM hind paw withdrawal threshold (g).

### DETAILED DESCRIPTION OF THE INVENTION

The present invention relies on the protective effect of acute inflammatory responses against the development of chronic pain. This effect was identified as a result of using transcriptome-wide data to investigate the molecular pathophysiological mechanisms in peripheral blood immune cells at the transcriptome-wide level that underlie the transition of acute to chronic low back pain (LBP). This finding was replicated in an independent cohort of patients with another musculoskeletal pain condition, temporomandibular disorder (TMD). Finally, rodent pain models were employed to elucidate the mechanism mediating the transition from acute to chronic pain.

The initial bioinformatics results indicated that there was a substantial difference in the time courses of transcriptomic changes in subjects with resolved pain compared to those with persistent pain. The trajectories show substantial differences: in the resolved pain group, several thousand genes were found to be differentially expressed over time, whereas there were no differences in the persistent pain group. Thus, the data herein support the notion that active biological processes protect from transitioning to chronic pain after an acute pain episode.

To identify the initial processes that drive these differences in trajectories between the resolved and the persistent pain groups at the gene level, functionally related sets of genes were compared at the pathway level. Neutrophil activation-dependent elevation of the inflammatory response at the acute stage of pain was found in subjects with resolved pain, which was decreased by the time of a follow-up visit (second visit). Conversely, subjects with persistent pain did not show any changes in their inflammatory response. These findings were replicated in an independent cohort for temporomandibular disorders (TMD). This was based on the rationale that there is a shared pathophysiology between different chronic pain conditions, as has been argued through both high clinical comorbidity and shared genetic heritability (Diatchenko et al., 2013; Meloto et al., 2018). The replication of the present findings in the TMD cohort corroborate the broad applicability of the present findings to other chronic pain conditions.

Interestingly, herein, no pathways with negative correlation between the resolved and the persistent pain groups were identified; in fact, the two pain groups showed strongly correlated processes. Instead, the difference between the two groups was highlighted in the magnitude of the regression slope, again supporting the notion that the resolved pain group's response intensity was substantially larger than that of the persistent pain group. These results were in line with the observed differences in the number of differentially expressed genes over time between the groups, and re-emphasized the (surprising) concept that an active biological process underlies pain resolution rather than pain progression to chronic status. The present results support the concept that this process is impaired in those who do not resolve acute pain over time and indicate time stratification of a cascade of processes resulting in a return to a normal, no-pain state (Serhan and Savill, 2005)—in a fashion similar to timely processes involved in wound healing (St Laurent et al., 2017; Seifert et al., 2012). The present findings are in line with the observation that the beginning of the inflammatory process programs its resolution (Serhand and Savill, 2005), and it is thus the failure to initiate an appropriate inflammatory response that may lead to chronic pain. This notion was further illustrated by the TMD cohort herein, which provided the additional advantage of the availability of a control no-pain group. Sharp upregulation of neutrophil-related inflammatory response at the acute stage of the TMD pain-persisting group but not the TMD pain-resolving group could be confirmed, and higher inflammatory states of chronic pain patients.

Using three different assays of prolonged but resolving pain in the mouse, it was confirmed that the acute treatment of inflammation with either the steroid, dexamethasone, or the NSAID diclofenac—although both effectively reducing pain behavior during their administration—greatly prolonged the resolution of neuropathic, myofascial, and especially inflammatory pain states. Three analgesics without anti-inflammatory properties (gabapentin, morphine, and lidocaine) produced short-term analgesic effects without affecting the overall duration of the painful (allodynic) episode. Further, the neutrophil-dependence of these effects was shown, with steroid-like pain prolongation being produced by neutrophil depletion and a complete blockade of allodynia produced by peripheral injection of neutrophils themselves. Furthermore, the mouse data herein confirmed the important roles of two neutrophil-specific proteins identified via human transcriptomics data, the alarmin proteins S100A8 and S100A9.
a. Taken altogether, the present results show that active immune processes confer adaptation at the acute pain state, and impairment of such inflammatory responses in subjects with acute LBP (or TMD) increases the risk of developing chronic pain. These adaptive inflammatory responses are intrinsically transcriptionally driven, probably modified by both genetics and environmental factors, and they can be inhibited by steroids and NSAIDs. Importantly, these responses are transient, which is probably the main reason they were previously overlooked. These conclusions may be expected to have a substantial impact on medical treatment of the most common presenting complaints to health care professionals. Specifically, the data herein shed a new light on the anti-inflammatory drugs in the treatment of acute LBP and likely other pain conditions, in particular regarding their long-term effects.

In the light of the above, the present invention allows the prevention and/or treatment of chronic pain by eliciting, sustaining potentiating or preserving an inflammatory process, thus solving the problem underlying the present invention. It is one finding underlying the present invention that such an inflammatory process may be transient, but is critical for pain resolution. Said inflammatory process may be present during a phase of acute pain or during a phase of chronic pain.

Generally, subjects with an insufficient inflammatory process (which may be e.g. intrinsic or induced) are at risk of chronic pain. When administering an analgesic in order to treat acute pain, it is thus beneficial if said analgesic elicits substantially no anti-inflammatory effect at the amount administered. For instance, an analgesic having no, or substantially no, anti-inflammatory properties may be used. In this way, inducing insufficiency of the inflammatory process is avoided. Another option is to elicit, sustain or potentiate an inflammatory process when administering an analgesic (e.g. simultaneously or prior to or after administering the analgesic). This may be done by administering a combination of an analgesic (whether per se with or without anti-inflammatory properties) and one or more of a pro-inflammatory compound, myeloid leukocyte cells and a substance that increases the number or activity of myeloid leukocyte cells. This option will mean that any anti-inflammatory effect of the analgesic can be counteracted. This is also the basis for the kit according to the invention, which comprises a combination of an analgesic with a pro-inflammatory compound, myeloid leukocyte cells and/or a substance that increases the number or activity of myeloid leukocyte cells.

The status of a subject's inflammatory processes plays an important role for both diagnosis and choice of treatment. For example, if the amount of inflammation markers in the blood of a subject suffering from acute or chronic pain is too low, this may have two consequences. Firstly, this is one reason why the subject's inflammatory process may be insufficient. Secondly, is an indication that the chronification of this pain should be prevented or treated, respectively, in particular by eliciting, sustaining or potentiating an inflammatory process. Additionally, it may be beneficial to determine whether such an inflammatory process may be elicited, sustained or potentiated to a sufficient degree. This may be determined by using a substance that activates neutrophils and comparing the degree of activity of neutrophils isolated from said subject prior to and after being contacted with said substance. If the neutrophils can indeed be activated, this is again an indication that the chronification of this pain should be prevented or treated, respectively, in particular by eliciting, sustaining or potentiating an inflammatory process.

The findings of present invention also allow the production an animal model of chronic pain. Such a model can be used as an assay for chronic pain and the respective treatments. It allows modulating inter alia the inflammatory response and/or the activity of neutrophils or other myeloid leukocyte cells in the animal. The basis for such a model is the discovery that an anti-inflammatory compound, or a substance or treatment that decreases the number or activity of myeloid leukocyte cells, promotes chronification of pain. Such a model also allows modulating the pain state of the animal by eliciting, sustaining potentiating or preserving an inflammatory process in the animal.

As used herein, the term “pro-inflammatory compound” refers to a compound that may have a direct pro-inflammatory effect and/or cause the production of another compound that has a direct or indirect pro-inflammatory effect. A preferred pro-inflammatory compound is selected from neutrophil-derived factors. More preferably, it is selected from the group consisting of an alarmin, a cytokine, a growth factor, a prostaglandin receptor agonist and combinations thereof. A preferred alarmin is selected from the group consisting of S100A8 and S100A9. A preferred cytokine is selected from the group consisting of IL-1α, IL-1β, IL-6, IL-8, TNF-α, IFN-γ, IL-12 and IL-18. Preferably, the growth factor is selected from the group consisting of EREG, VEGF and GM-CSF. A preferred prostaglandin receptor agonist is selected from the group consisting of a PTGIR agonist and a PTGER4 agonist.

As used herein, the term “myeloid leukocyte cells” includes, but is not limited to, dendritic cells, monocytes, macrophages and granulocytes. This term refers to myeloid immune cells. Preferred myeloid leukocyte cells are granulocytes, monocytes and macrophages. Granulocytes include neutrophils, basophils and eosinophils, with neutrophils being preferred. Thus, particularly preferred myeloid leukocyte cells are neutrophils, monocytes and macrophages. The most preferred myeloid leukocyte cells are neutrophils. A different way of describing myeloid leukocyte cells is by distinguishing between neutrophils on the one hand and the group of myeloid leukocyte cells except neutrophils on the other hand (which group includes dendritic cells, monocytes, macrophages, basophils and eosinophils, with monocytes and macrophages being its preferred members). For administration to a subject, it is preferred that myeloid leukocyte cells, preferably neutrophils, are purified. Preferably, a number of 5×10⁵ to 5×10⁸, preferably 5×10⁶ to 5×10⁷ corresponding cells are administered. The cells are preferably injected locally.

As used herein, the term “subject” preferably refers to a mammal, for example a dog, a cat, a horse, a camel or a human. The most preferred subject is a human.

As used herein, a “transient” inflammatory process may last for example 4, 6, 8, 10, 12, 14, 16, 18, 20 days or more than 20 days.

As used herein, “chronic pain” preferably relates to pain that is experienced by a subject for more than 6 months and more preferably for more than 3 months. Alternatively, it may refer to pain that extends beyond the expected period of healing, or to pain that has no biological value and persists past normal tissue healing. In the context of the present invention, chronic pain is preferably a neuropathic pain state, a myofascial pain state, an inflammatory pain state, chronic postoperative pain, low back pain or temporomandibular disorder.

As used herein “acute pain” preferably refers to pain that is experienced by a subject for less than 30 days.

As used herein, “alarmin” preferably refers to S100A8 and/or S100A9. Each alarmin is preferably administered at a dose of 0.1 to 1000 μg/day, more preferably 1 to 100 μg/day or 10 to 100 μg/day. An alarmin is preferably injected locally.

### EXAMPLES

In a first step, a study was designed and implemented to identify cellular and molecular mechanisms underlying acute-to-chronic pain transition in humans, using data from a cohort of subjects with low back pain (LBP).

### Example 1: Study Design in the LBP Cohort

The human LBP cohort is part of a larger study, PainOMICs, registered on clinicaltrials.gov (NCT02037763) and funded by the European Community in the Seventh Framework Programme (FP7)—THEME (HEALTH.2013.2.2.1-5—Understanding and controlling pain) to evaluate biomarkers related to pain. The protocol was approved by the Ethical Committee at the University Hospital of Parma (protocol number 43543 version 8). All patients signed a written informed consent before the enrolment and were followed up for one year.
a. The primary objective of this study was to investigate associations between genome-wide transcriptomics and the development of persistent chronic LBP, in patients developing persistent chronic pain symptoms three months after an episode of acute LBP. Subjects enrolled in this study were a part of a larger protocol that follows the same design. For the current study, retrospectively the first 50 patients with resolved pain and the first 50 patients with persistent pain were selected. The patients were all Caucasian adults.
b. Sample sizes were not estimated due to the hypothesis-free approach taken here, but the LBP cohort herein is similarly sized to other human transcriptomics studies in pain identified group differences (Dorsey et al., 2019; Bratus-Neuenschwander et al., 2018; Guo et al., 2017; Held et al., 2019; Theken et al., 2019).
c. The criteria for enrollment was the presence of acute LBP; that is, pain between the costal margins and the gluteal fold. All patients were evaluated using a Numeric Rating Scale (NRS), a scale to assess pain from 0 to 10, where 0 is “no pain” and 10 is “and “worst pain imaginable” and the Pain Detect Questionnaire (Freynhagen et al., 2006), to evaluate the neuropathic pain component. Inclusion criteria included a back pain level of ≥4 on the NRS, with a duration of no more than six weeks prior to the first visit, thus defining an acute phase. Exclusion criteria were history (in the last 6 months) of persistent chronic or acute LBP episodes, recent history (<1 year) of spinal fracture, pain in the back due to spinal tumor or infection, evidence of clinically unstable disease, severe psychiatric disorder (excluding mild depression) or mental impairment, and pregnancy.
d. Each patient was classified by clinical evaluation and diagnostic blocks, to determine the pain generator using these six classes: facet joint pain, sacroiliac pain, discogenic pain, spinal stenosis, back pain with predominant radiculopathy, and non-specific low back pain (Allegri et al., 2016). Patients were medically assessed and treated accordingly to physician's decision, independently of the protocol. Drugs used and medical history were also recorded.
e. Clinicians followed a standardized protocol for treating acute LBP with drugs addressed to reduce acute inflammatory flare (NSAIDs and systemic steroids) plus opioids if the pain was severe and/or if it had a greater impairment on daily activity. The choice of drugs was driven only by clinical signs and not by any measures of inflammatory flare. Thus, all patients had the same treatment.

Patients were seen at two time points: at the time of enrollment (t₀), and at a follow-up visit (t₁) three months later. The resolved pain group (R) patients were defined as those who self-reported day-averaged pain of less than 4 on the NRS in the week before the follow-up visit; those reporting levels of 4 or higher were defined as the persistent pain group (P). The value of 4 was previously defined as an optimal cut point for “clinically significant” pain (Hirschfeld and Zernikow, 2013; Shafshak and Elnemr, 2020) and in clinical practice, this cut off is used to decide if pain may lead to functional and clinical disability and thus should be treated. The researcher who performed the laboratory analysis was masked about the group of patient analyzed.

### Example 2: Blood Collection and RNA Extraction in the LBP Cohort

An RNA standard operating procedure was developed and validated to achieve uniformity and provide details in each sample (Dagostino et al., 2017). Whole peripheral blood was collected at both visits, t0 and t1, at the Pain Service of University Hospital of Parma. Tempus blood RNA tubes were used (Applied Biosystems, n. 4342792, Beverly, MA, USA), shaken vigorously for 10-15 sec immediately after collection for RNA stabilization, and stored at −20° C. Total RNA was isolated using Maxwell® 16 LEV simplyRNA Blood Kit (Promega, Madison, WI, USA) according to the manufacturer's instructions. After, total RNA was quantified using the NanoDrop ND-1000 (Thermo Scientific, Waltham, MA, USA), and optical purity of RNA was defined according to the 260/280 ratio not less than 1.8 and 260/230 ratio between 1.8-2.2 of the isolated RNA. Then, the RNA integrity was assessed with the Agilent 4200 TapeStation (Agilent Technologies, Santa Clara, CA, USA) using the RNA Screen Tape assay (Agilent Technologies, n. 5067-5576). An average of 8 μg of total RNA was recovered from frozen whole blood, displaying RIN values between 6.5-8.4. Purified RNA was stored at −80° C. in 25-μl aliquots.

### Example 3: Whole Transcriptome Sequencing (RNA-Seq) in the LBP Cohort

All RNA-sequencing was performed by the Genome Quebec Innovation Centre (McGill University, Montreal, Canada). Transcriptome libraries were generated from 1 μg of total RNA using the Kapa RNA-stranded Sample Prep Kit (KK8400, KAPABiosystems, Wilmington, MA, USA) following the manufacturer's protocols. Briefly, poly-A mRNA was purified using poly-T oligo-attached magnetic beads using two rounds of purification. During the second elution of the poly-A RNA, the RNA was fragmented and primed for cDNA synthesis. During cDNA synthesis, dUTP was incorporated in the second-strand synthesis, and subsequently the dUTP-containing strand was selectively degraded. Adenylation of the 3′ ends and ligation of adapters were done following the manufacturer's protocol. Enrichment of DNA fragments with adapter molecules on both ends was performed using 10 cycles of PCR amplification using the KAPA PCR mix and Illumina-adapted primers cocktail. Paired-end 2×100 nucleotides sequencing was performed using the Illumina HiSeq2000 machine running TruSeq v3 chemistry at Genome Québec.

### Example 4: Processing of RNA-Seq Data in the LBP Cohort

Deep-sequencing reads were aligned on the human genome version hg19/GRCh37 using STAR aligner version 2.5.1b (Dobin et al., 2013). Gene-level counting was performed using featureCounts version 1.6.0 (Liao et al., 2014), using RefSeq gene boundaries (O'Leary et al., 2016), Gene-centric outlier expression levels detected using an iterative leave-one-out method (George et al., 2015).

The quality of sequencing was confirmed by the RNA integrity numbers (RIN; mean=7.9, SD=0.8), total average number of sequenced paired-end reads (mean=135.8M, SD=26.6M, min=49.0M), sequencing mapping rates or properly paired mapped reads to total paired reads in sequencing (mean=95.8%, SD=0.8%, min=93.8%), and by the stability of results from DESeq2 after removing samples with highest gene expression outliers (54). Following principal component analyses of the transcriptomes, it was established that the variables associated with the first few principal components considered in differential gene expression analyses were RIN, age, sex, and smoking status. In total, 196 time-paired RNA-Seq samples for 98 individuals were obtained.

Finally, to enable comparisons between different contrasts and to correct for multiple testing from gene pools of the same sizes, the counts on genes were transformed into units of transcripts per million (tpM) for each sample; it was required that all samples featured tpM levels of at least 0.1, hence only 12081 genes out of the 27937 total (43.2%) were considered for further analyses. Resulting DESeq2 P-values obtained using counts (not tpM) were re-corrected for multiple testing with FDR, this time from the shortened list of genes.

### Example 5: Pathway Analyses in the LBP Cohort

A pathway enrichment score was computed using the R package ‘fgsea’ (48) with gene sets from Gene Ontology's biological processes (The_Gene_Ontology_Consortium, 2019; Ashburner et al., 2000). The pathways' definition files were taken from the URL http://download.baderlab.org/EM_Genesets/December_01_2019/Human/symbol/GO/; December 2019 version. The assignment of activation or repression of a pathway was based on the accumulated evidence of concordant fold-change direction of genes that are part of the pathway (Subramanian et al., 2005); a positive (negative) value indicates that many or most genes of the pathways were regulated in an up (down)-ward fashion. The genes with the greatest difference in expression in a pathway were defined as “leading edge” genes, as they determined in which direction the pathway was anticipated to be regulated. Here, a differentially expressed pathway could reach statistical significance even though few or none of the associated genes individually reached statistical significance for differential expression in a transcriptome-wide analysis.

### Example 6: Blood Cell Type Population Fraction Estimates in the LBP Cohort

Estimates of blood cell type population fractions from RNA-Seq data were obtained using CIBERSORT (Newman et al., 2015). Input data for CIBERSORT were gene expression levels, in tpM units. tpM were derived from featureCounts (Liao et al., 2014), counts on genes normalized to gene lengths provided by RefSeq (O'Leary et al., 2016). Estimates for cell type fractions were used as co-variables for some analyses of differential expression of genes, accounting for different populations of cell types for those with fractions ≥1%.

### Example 7: Blood Cell Type Gene Expression in the LBP Cohort

Gene lists for blood cell types were extracted from the LM22 gene expression matrix of CIBERSORT (Newman et al., 2015). A gene was retained in a cell's list only if its expression level was greater than the average across all other cell types. The gene overlap between four selected cell types was calculated using the online tool to draw a 4-way Venn diagram at interactivenn.net (Heberle et al., 2015).

### Example 8: Differential Gene Expression in the LBP Cohort

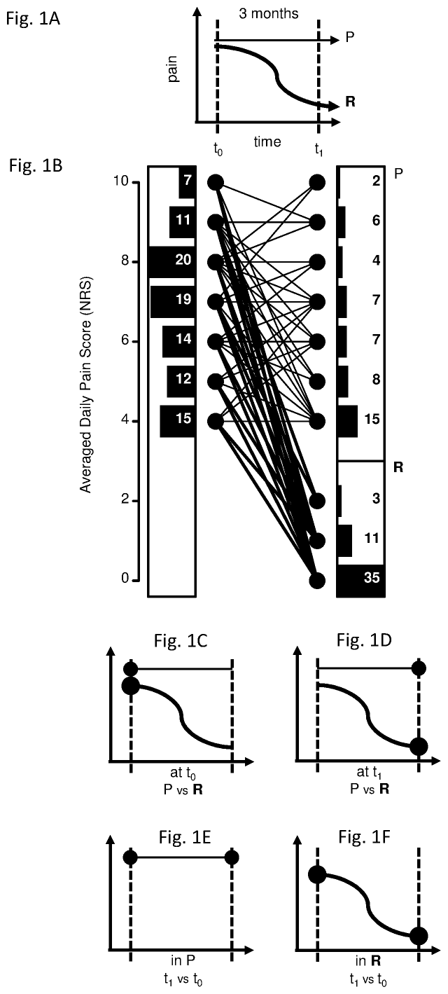

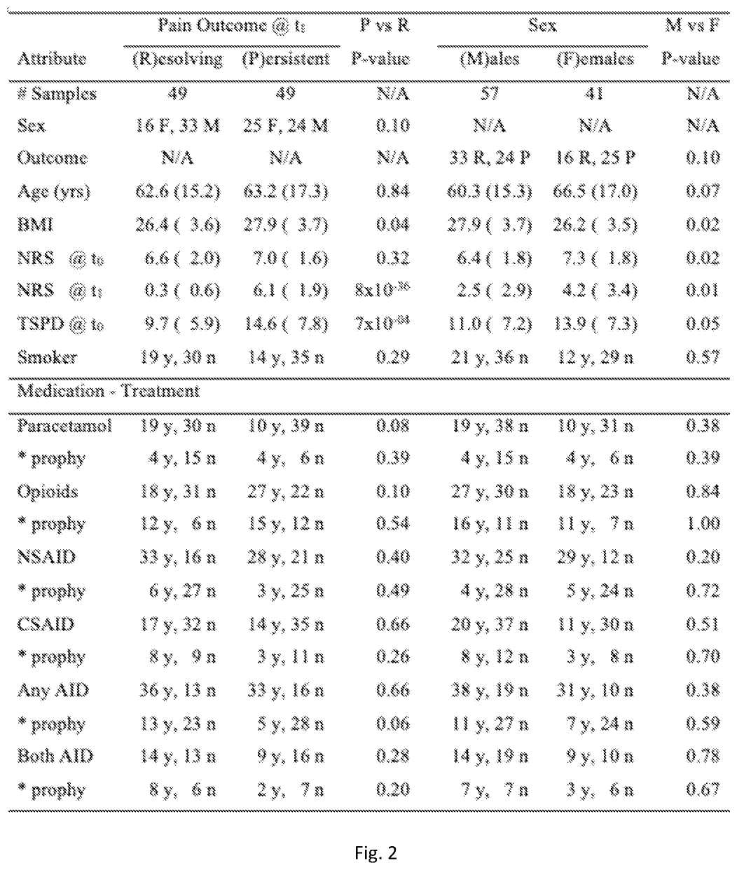

Genome-wide transcriptomics were assessed in the cohort of 98 patients with LBP at the acute episode (t₀), and a follow-up visit (t₁) three months later. Study design, demographics, and patient characteristics are presented in FIG. 1A-B, and FIG. 2. Selected patients reported substantial, self-declared pain (0-10 Numeric Rating Scale; NRS) in their lower back at enrollment (to: mean=6.8; SD=1.8; min=4; max=10), but a much broader pain spectrum was observed at follow-up (t₁: mean=3.2; SD=3.2; min=0; max=10). Study participants were dichotomized into two groups based on their pain scores at the second visit: those with resolved pain (“R”; N=49) and those with persistent pain (“P”; N=49) (FIG. 1B). Importantly, no differences were found for the pain outcome with drug classes consumed either between the clinical visits or used prophylactically (FIG. 2).

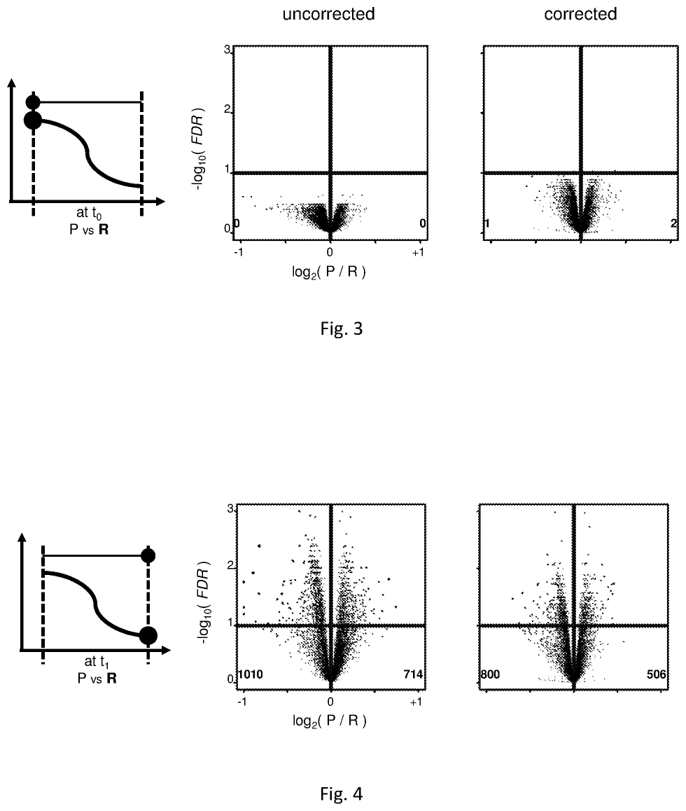

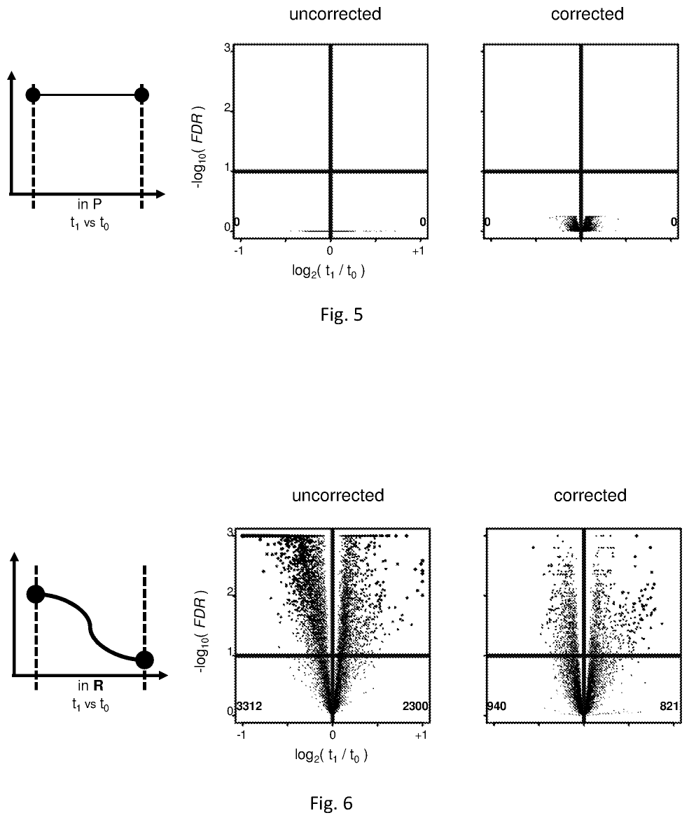

Then, differences between patient groups at the transcriptomics-wide level were tested. At the first visit, there were no differentially expressed genes that reached genome-wide statistical significance between R and P patients (FIG. 3, column “uncorrected”). By the time of the second visit, more than 1700 differentially expressed genes were detected at the genome-wide scale between R and P patients (FIG. 4). When time trajectories were considered, contrasting gene expression between the two visits in P patients identified no differentially expressed genes (FIG. 5), whereas in R patients, more than 5500 genes were differentially expressed (FIG. 6). This pattern remained after controlling for blood cell type abundances, when analyses of differential expression of genes were repeated using estimated fractions of cell type populations as additional co-variables (Newman et al., 2015). A total of 1700 genes remained differentially expressed in those with resolved pain, while in those with persistent pain there were still no changes (FIG. 3-6, column “corrected”).

Together, the genome-wide transcriptomics analyses herein indicate that the subjects who resolved pain overtime have abundance of active biological processes underlying recovery and these processes partially driven by changes in blood cell composition.

### Example 9: Differential Blood Cell Type Populations

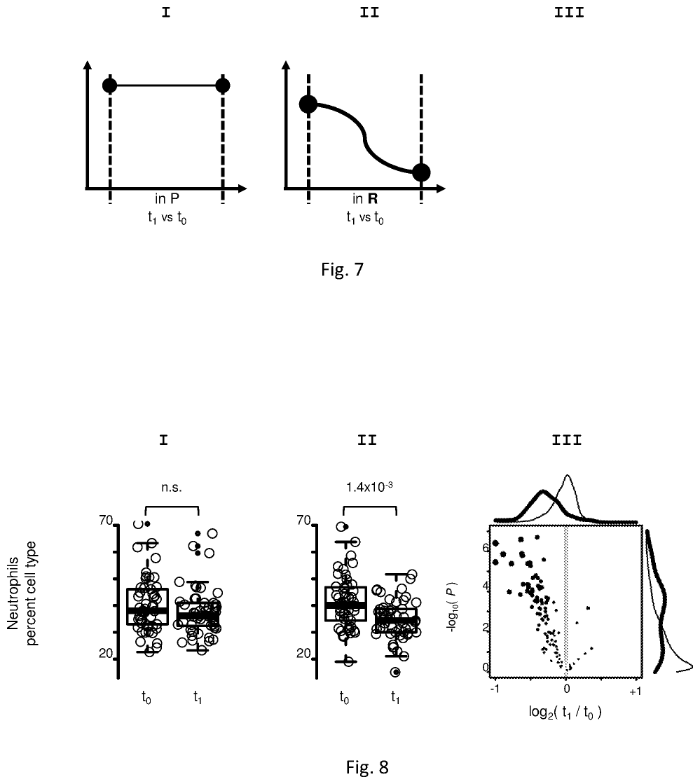

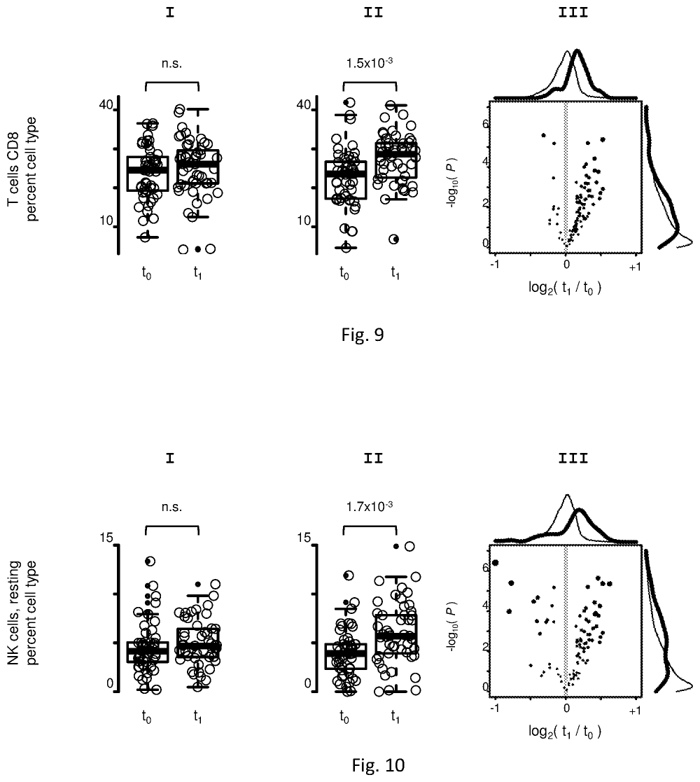

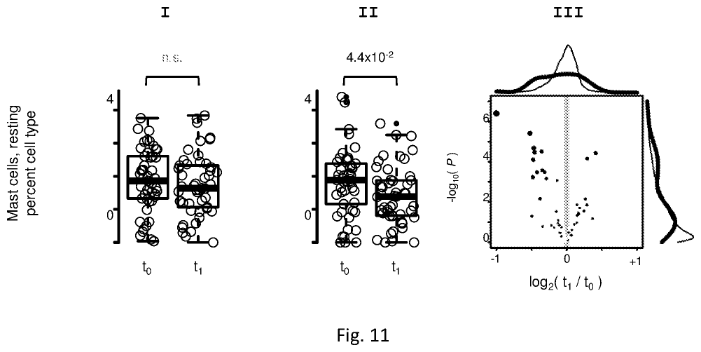

Next, the changes in relative cell type population fractions were estimated from the multiplexed RNA sequencing experiments (Newman et al., 2015), tracking cell type population changes in different contrasts (FIG. 7). In P participants, no changes in blood cell type populations were detected over time (FIG. 8-11, column I). However, in R patients statistically significant differences were found between the two visits in four cell types, with the largest difference observed in the numbers of neutrophils decreasing over time (P=1.4×10⁻³) (FIG. 8). The other significant differences included increased number of CD8+ T cells (P=1.5×10⁻³) (FIG. 9), resting natural killer (NK) cells (P=1.7×10⁻³) (FIG. 10), and decreased number of resting mast cells (P=4.4×10⁻²) (FIG. 11).

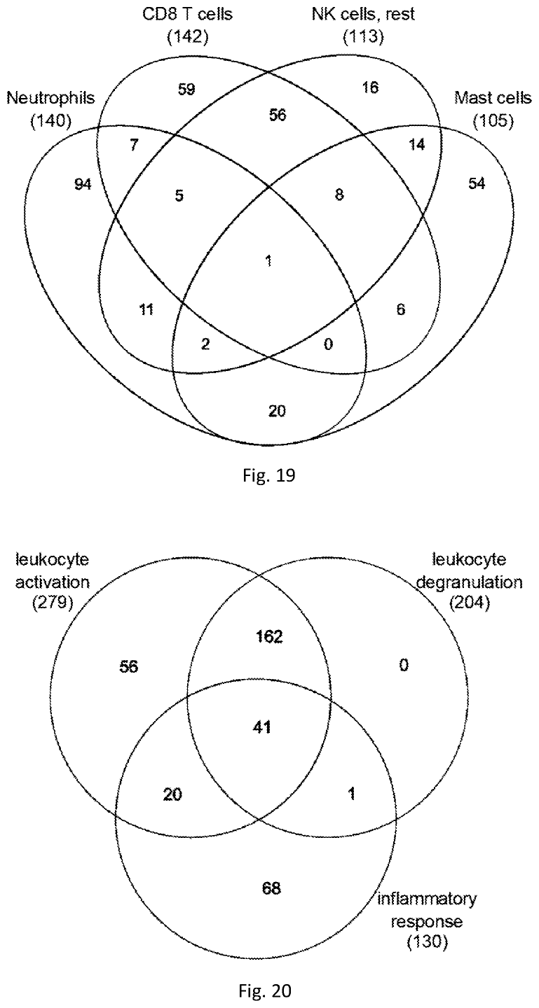

Also, a list of genes expressed by each cell type was built using CIBERSORT's LM22 “pure” cell type expression matrix. A gene was retained in the list if the expression level in that cell type was greater than the average across all other cell types (FIG. 19). These genes are highlighted for differential expression in the volcano plots (FIG. 8-11, column III). Thus, changes in cell type fractions can be tracked down to changes in gene expression, with matching change directions between fraction estimates and gene levels, with, again, the largest, consistent, and most substantial contribution from neutrophil-specific genes down-regulated between visit t₀ and t₁.

### Example 10: Results of Pathway Analyses

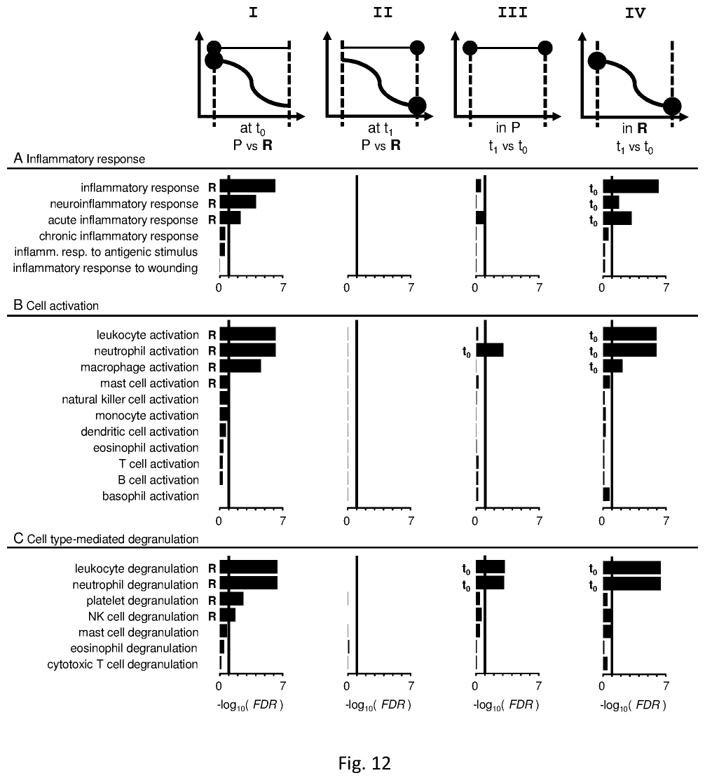

Next, biological pathways were analyzed instead of individual differentially expressed genes (FIG. 12). Many biological pathways differentially expressed at a genome-wide level were found even in the comparisons where no individual genome-wide significant differentially expressed genes were identified, which can occur when a substantial amount of genes of the same pathway change expression in the same direction. Since it was observed that active transcriptional processes underlined pain recovery over time, a focus was placed on biological pathways at acute stage that will yield the follow-up changes.

At the first visit (t₀), the most differentially expressed pathways were related to cell activation and immune responses, and they were elevated in the R group. These processes seemed to be driven by neutrophil activation and degranulation, and by elevated inflammatory response. Even though some of the leading-edge genes are shared between these two pathways, for the most part they describe two different biological processes (FIG. 20). This unexpected enhanced inflammatory response in R participants was consistently observed for other related inflammatory pathways (FIG. 12A, column I). With time, there were barely any changes in the inflammatory response pathways in the P group (FIG. 12A, column III). However, in the R group, the inflammatory response pathways were convincingly down-regulated over time, at t₁ compared to to (FIG. 12A, column IV).

The enhanced inflammatory pathways seemed to be driven by neutrophil activation through degranulation. Leukocyte activation and degranulation pathways were found noticeably activated at to in R patients. Among leukocytes, neutrophils were the most activated, followed by macrophages and mast cells (FIG. 12B, column I). Degranulation of neutrophils showed largest changes compared to platelet or natural killer cells (FIG. 12C, column I). Neutrophil activation and degranulation pathways were decreasing with time in both pain groups, though this decrease was more noticeable in R compared to P patients (FIG. 12). Within the top hits SLC11A1 was found, a divalent metal transporter that is specific to myelo-monocytic cells including neutrophils, monocytes and macrophages (Cellier et al., 1997; Canonne-Hergaux et al., 2002), and S100A8 and S100A9—neutrophil-specific genes coding for calcium-binding “alarmins” critical in the development and regulation of inflammation, which are known to comprise approximately 45% of the cytoplasmic proteins in neutrophils and function as homo- or heterodimers (Wang et al., 2018).

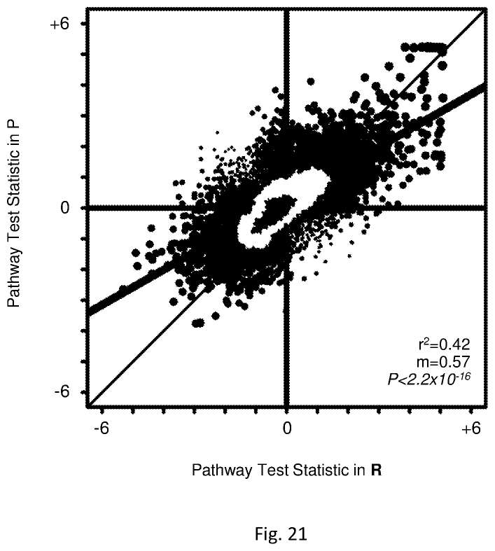

A positive correlation in transcriptional changes over time was found between R and P patients (FIG. 21), indicating that all individuals displayed similar biological responses and pathways, regardless of the pain outcome at the second visit (slope=+0.57, P<2.2×10⁻¹⁶, r=42%). The difference between groups was in the magnitude of the response: the R group response intensity was about 75% larger than that of the P group.

### Example 11: Temporomandibular Disorders (TMD) Replication Cohort

Reproducibility of the LBP results was investigated in two replication cohorts from a study of painful temporomandibular disorder (TMD), which is is another musculoskeletal pain condition. The cohorts were community-based volunteers from four U.S. study sites recruited into the “Orofacial Pain Prospective Evaluation and Risk Assessment” (OPPERA) study that began in 2006 (Slade et al., 2016). The first replication cohort comprised adults who had first-onset, acute TMD when enrolled from 2013-2016, and who were followed for a six months period to identify those who had persistent TMD. The cohort was selected during screening interviews of 166,062 phone numbers between 2013 through 2016, which identified 327 subjects who reported ≥5 days of TMD pain in the preceding 30 days, but no TMD symptoms at any time in their life before that period. During a baseline (t0) study visit, 162 of them has examiner-classified TMD based on the DC-TMD criteria. Six months later (t1), 118 were again examined to classify their clinical TMD status. The TMD subjects who no longer met TMD criteria at 6 months were defined as the resolved pain group (R), and otherwise as the persistent pain group (P). In total, 64 prospective samples were analyzed in that prospective cohort. The second TMD cohort was from another OPPERA study protocol that enrolled adults into a cross-sectional from 2014-2016 (61). This analysis used samples from 86 adults with examiner-classified chronic TMD based on DC-TMD criteria and 65 TMD-free controls.

At each visit, participants donated blood, which was collected in PaxGene tubes and stored at −80° C., and RNA isolated using Qiacube column-based separation or Chemagic magnetic bead separation. Approximately 250 ng Total RNA was used for mRNA-Seq library preparation by following the Illumina TruSeq stranded mRNA sample preparation guide (Illumina Inc, San Diego, CA). The first step in the workflow involved purifying the poly-A containing mRNA molecules using poly-T oligo-attached magnetic beads. Following purification, the mRNA was fragmented into small pieces using divalent cautions under elevated temperature. The cleaved RNA fragments were copied into first strand cDNA using reverse transcriptase and random primers. This was followed by second strand cDNA synthesis using DNA Polymerase I and RNase H. Strand specificity was achieved by replacing dTTP with dUTP in the Second Strand Marking Mix (SMM), as the incorporation of dUTP in second strand synthesis effectively quenched the second strand during amplification. Further specificity was achieved by addition of Actinomycin D to the First Strand Master Mix Act D (FSA), which prevented spurious DNA dependent synthesis during first strand synthesis, while allowing RNA dependent synthesis. The resulting cDNA fragments then went through an end repair process, the addition of a single ‘A’ base, and then ligation of the adapters. The products were then purified and enriched with PCR to create the final RNA-Seq library. After RNA-Seq libraries were subjected to quantification process, pooled for cBot amplification and subsequent sequencing run with Illumina HiSeq 3000 platform with 50 bp single read sequencing. After the sequencing run, demultiplexing with Bcl2fastq2 was employed to generate the fastq file for each sample.

In total, 65 healthy controls samples, 86 samples from chronic TMD cases, and 64 prospective samples were analyzed, in which 42 samples were from subjects with acute pain at t0, from whose there were 14 follow up samples from subjects with persistent pain and 8 from subjects who resolved their pain at t1.

### Example 12: Replication of Findings in the Independent TMD Cohort

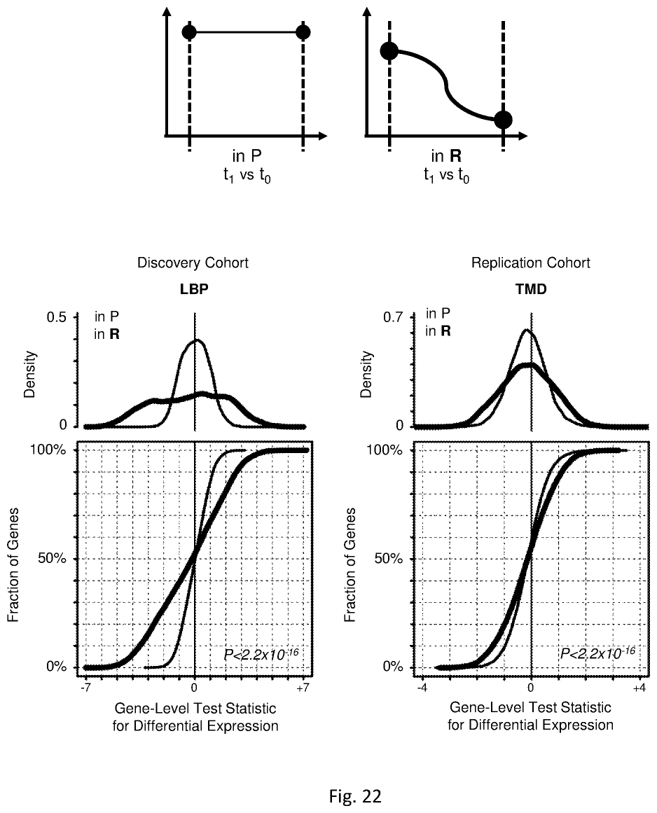

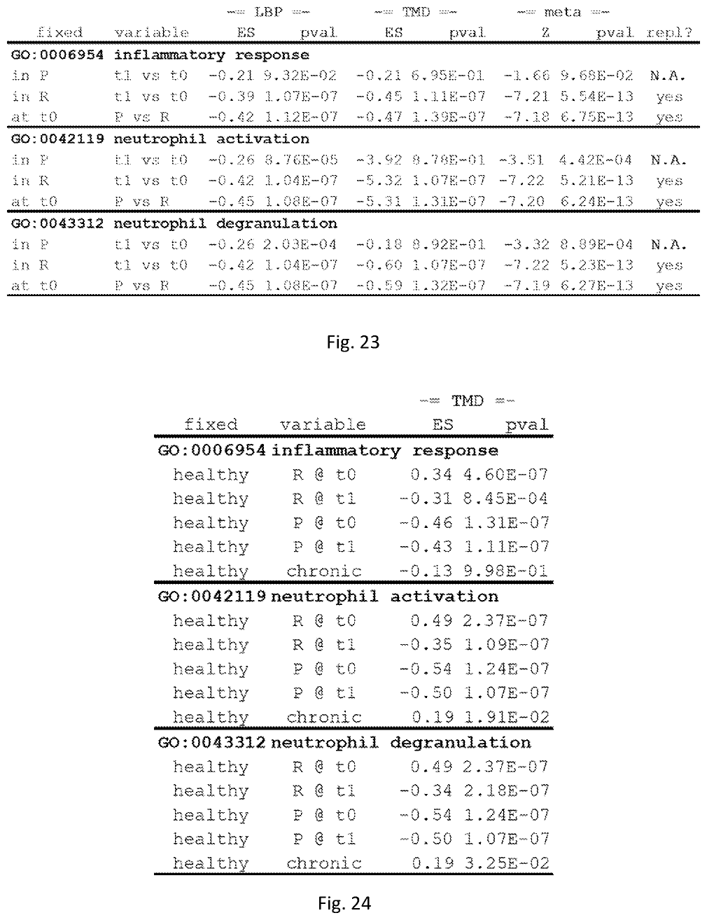

The findings were replicated using a prospective cohort of similar design (FIG. 22, top). The replication cohort comprised subjects with temporomandibular disorder (TMD). Although the pathophysiology of TMD is likely not identical to LBP, it was hypothesized that the active contribution of the immune system in the transition to chronic pain could be shared. At the first visit, all subjects displayed acute symptoms of TMD, whereas at a second visit, some subjects had their pain resolved (R) and in others pain persisted (P). In this cohort as well, a larger number of differentially expressed genes in subjects in the R group than in the P group was observed (FIG. 22, bottom), and elevated activity of inflammatory, neutrophils activation and degranulation pathways in the R group (FIG. 23). From CIBERSORT's gene expression input matrix, one hundred genes were identified whose expression in neutrophils is greater than the average across all other cell types. At to, 80% of these genes were found to be more expressed in the R group in both LBP and TMD cohorts, and about 75% of these genes to be more expressed at t₀ than t₁ in the resolved pain group, in both LBP and TMD cohorts.

Additionally, the TMD cohort allowed comparison with healthy controls and patients with chronic TMD (FIG. 24). In comparison with healthy controls, the R group displayed a significantly higher inflammatory response at the acute stage (fgsea pathway Enrichment Score ES=+0.32>0, P=1.1×10⁻⁵), whereas the P group displayed a significantly lower response (ES=−0.32<0, P=1.3×10⁻⁷). By the time of the second visit, each pain group displayed a significantly reduced inflammatory response compared to the healthy group (P=3.1×10⁻⁴ in R, P=1.1×10⁻⁷ in P). The same pattern was observed for neutrophil activation and degranulation pathways.

Also upregulated neutrophil activation and degranulation pathways were observed in subjects with chronic TMD in comparison with healthy controls (FIG. 24, ES=+0.19>0, P=1.9×10⁻² and 3.3×10⁻², respectively), although to a lesser degree than for the R group at to (ES=+34>0, P=4.6×10⁻⁷). These results indicate the importance of the upregulation of inflammatory response at the acute stage of musculoskeletal pain as a protective mechanism against the development of chronic pain.

### Example 13: Mouse Experiments

To assess how the inhibiting the inflammatory response would impact the resolution of pain from an acute injury a series of mouse experiments was performed using neuropathic and inflammatory assays, and a variety of anti-inflammatory and non-anti-inflammatory analgesics. In addition, the relevance of neutrophils was assessed directly. All mouse studies were performed by an experimenter blinded to drug condition, and subjects were randomized to drug groups. Power analyses were not performed because effect sizes could not be anticipated in advance. Instead, sample sizes typical for these assays were used (PMID: 16842916).

Mice: CD-1 male and female mice aged 6-12 weeks (ICR:Crl, Charles River, St. Constant, QC) were used in these experiments. All mice were housed in standard shoebox cages with 2-4 (same-sex) per cage in a light (14:10 h, lights on at 07:00 h) and temperature-controlled (20±1° C.) environment with ad libidum access to food (Harlan Teklad 8604) and tap water. Mice were acclimated to the vivarium for 7 days post-arrival and before testing. Each mouse was used in a single drug experiment. Equal numbers of male and female mice were included in each cohort.

Drugs: Dexamethasone (PromoCell, Heidelberg, Germany) was administered via daily subcutaneous injections at a dosage of 0.5 mg/kg/day. Diclofenac, morphine, and gabapentin were administered via intraperitoneal (i.p.) injection at a dose of 25 mg/kg/day, 5 mg/kg/day, and 100 mg/kg/day, respectively. Lidocaine was administered via subcutaneous (s.c.) injection of 20 μl of 2% lidocaine into the plantar hind paw. All drugs other than dexamethasone were purchased from Sigma-Aldrich (St. Louis, MO, USA). Anti-Ly6G (BioX Cell [InVivoPlus anti-mouse Ly6G/Ly6C (Gr-1)]) and its isotype control (BioX Cell [InVivoPlus rat IgG2b isotype control, anti-keyhole limpet hemocyanin]) were injected at a dose of 125 μg/day. S100A8 and S100A9 were injected (10 μl) into the plantar surface of the inflamed (see below) hind paw at a dose of 1 μg/day.

Neutrophils: Neutrophils were isolated using the EasySep Mouse Neutrophil Enrichment kit (Stem Cell Technologies, Vancouver, Canada) as per manufacturer's instructions. Briefly, blood was collected by cardiac puncture in a 21-gauge needle coated with 10% EDTA and pooled from 2-3 mice in PBS/2 mM EDTA. Red blood cells were lysed after incubation in 0.83% NH₄Cl for 10 min at 4° C., with the reaction stopped by adding PBS+10% FBS. Cells were centrifuged at 300 g for 5 min and resuspended in PBS+10% FBS. Cells were then incubated with rat serum and enrichment cocktail for 15 min at 4° C. and centrifuged at 300 g for 10 min. Cells were then resuspended in biotin selection cocktail for 15 min at 4° C., followed by magnetic beads for an additional 10 min, and placed into the separation magnet for 3 min. Non adherent cells were removed, and adherent cells washed off of the walls of the tube and resuspended in PBS for injection. Cells (5×10⁶, in 10 μl) were injected into the plantar surface of the inflamed hind paw (see below) of mice within 30 min of isolation.

Assays: The chronic constriction injury (CCI) procedure (Bennett and Xie, 1988) was performed under isoflurane anesthesia, and consisted of an incision made below the mouse's left hip bone followed by exposure of the sciatic nerve and the application of three ligatures with 4/0 silk thread loosely tied around the sciatic nerve proximal to its trifurcation. The incision was then closed in layers. Mice were given 6 days of recovery before behavioral testing commenced. This assay is a well known model of neuropathic pain.

A recently developed mouse assay of myofascial low back pain was used in one experiment (Porta and Tappe-Theodor, 2020). Nerve growth factor (NGF; Sigma; 0.75 μg dissolved in a 30 μl volume of phosphate-buffered saline) was injected twice, 4 days apart. Mice were lightly anesthetized under isoflurane/oxygen anesthesia and placed in a prone position. After shaving the low back skin, the L5 spinous process was located using the iliac crest as a landmark. Injections were made 1.5 mm lateral to the L5 spinous process using a 30-gauge needle attached to a 1-ml syringe into the muscle (1 mm up from the bone).

In most experiments, complete Freud's adjuvant (50% CFA, 20 μl volume) was injected into the plantar surface of the mouse's left hind paw as a model of inflammatory pain.

Before and after all injuries, mechanical paw-withdrawal threshold was measured with von Frey filaments using the up-down staircase method of Dixon (Chaplan et al., 1994), as previously described (Mogil et al., 2006). In every experiment, a baseline threshold determination was made on Day 1. Drugs or cells were administered daily from Day 0 to Day 6 (dexamethasone, diclofenac, gabapentin, lidocaine), on Day 3 and Day 5 (S100A8, S100A9, and neutrophils), or every other day from Day 0 to Day 20 (anti-Ly6G). von Frey testing on Day 6 occurred 1 hour after the drug injection on that day. Testing continued until both vehicle and drug groups had returned to their baseline statistically (with the exception of CFA+diclofenac), even though not all individual mice had necessarily recovered. Thus, in an attempt to minimize the duration that mice remained in pain, the length of experiments were different depending on the assay used and the purpose of the experiment, from 40 days post drug to 120 days post-drug.

Data analysis: Time course data of von Frey thresholds are shown graphically. To quantify the acute effects of drugs on mechanical thresholds during drug exposure (on Day 4-6 after CCI or CFA), the percentage of maximum possible allodynia was quantified as: % allodynia=[(baseline threshold−post-drug threshold)/baseline threshold]×100. A reduction of % allodynia would indicate acute analgesic and/or anti-inflammatory action of the treatment. To quantify the duration of the entire CCI- or CFA-induced allodynic episode, the day-by-day mechanical thresholds of each subject was considered. The Days to Return quantification provided consisted of the first of two consecutive days that a subject's threshold had returned to within 0.5 SD (via group means) of its baseline threshold. The use of 0.5 SD is, of course, arbitrary, but the use of other values yielded highly similar conclusions.

### Example 14: Impaired Inflammatory Response Prolongs Resolution of Painful Behavior in Mouse Preclinical Assays

The human transcriptomics results herein indicated that active inflammatory responses, particularly those regulated by neutrophils, contributed to pain resolution. It was hypothesized that inhibition of this active immune response will lead to the prolongation of pain, and designed experiments to test this hypothesis in mice using pain assays featuring evidence of pain that is persistent but of finite duration.

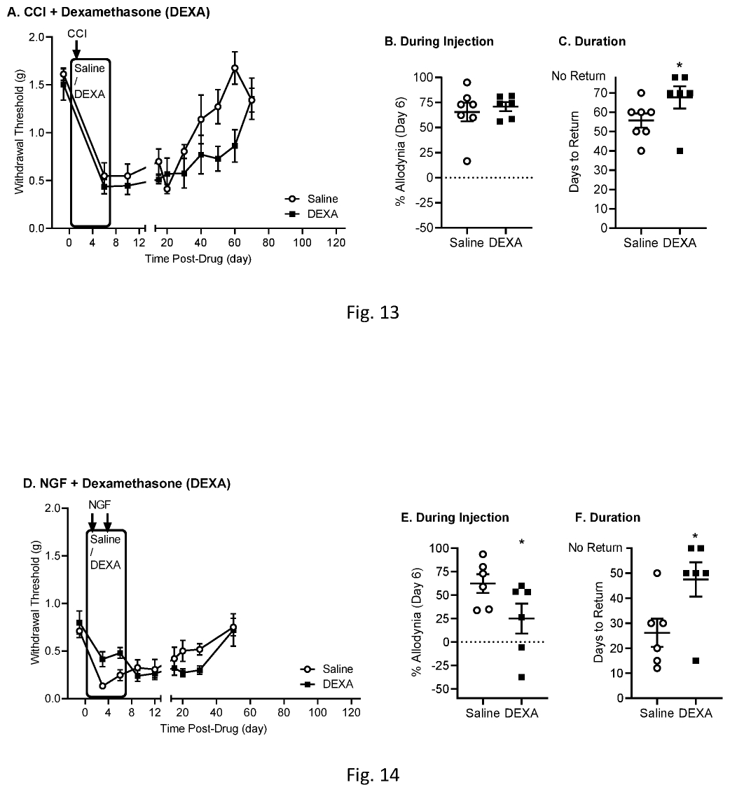

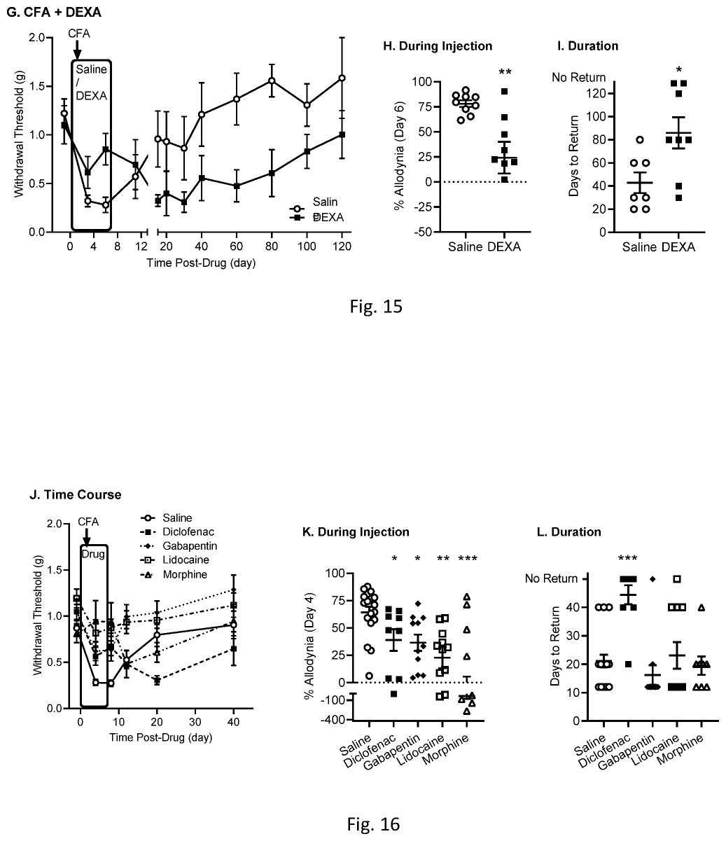

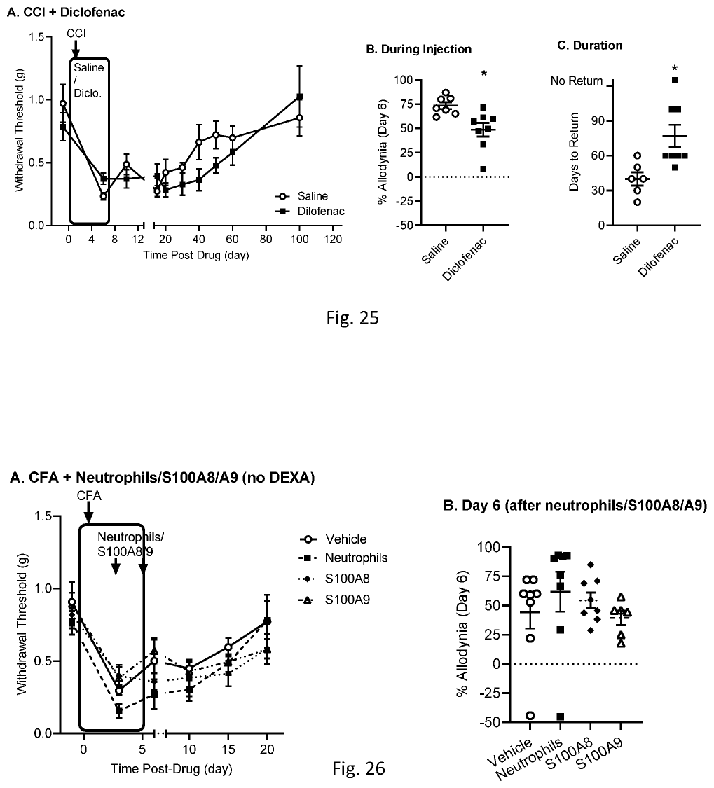

Initial experiments used the classic steroidal anti-inflammatory drug, dexamethasone. Mechanical pain sensitivity was assessed before and at multiple time points after chronic constriction injury (CCI) of the sciatic nerve, injection of nerve growth factor (NGF) into the muscles of the low back, or inflammatory injury using the cell-mediated immunity stimulator, complete Freund's adjuvant (CFA, inactivated Mycobacteria tuberculosis in oils). Dexamethasone or saline vehicle was administered daily for six days following CCI or CFA. All three injuries produced mechanical allodynia, that is hypersensitivity to the evoking mechanical stimulus, lasting approximately 30-60 days (depending on the assay) in saline-treated mice, respectively (FIG. 13, left, FIG. 14, left, FIG. 15, left). It was observed that the steroid had no effect on hind paw allodynia at the end of the treatment period after CCI (t₁₁=0.5, P=0.63; FIG. 13, middle), as might be expected given that CCI does not produce hind paw inflammation. By contrast, dexamethasone produced robust inhibition of allodynia from NGF (t₁₀=2.4, P=0.04; FIG. 14, middle) and CFA (t₁₃=3.0, P=0.009; FIG. 15, middle) on day 6 post-injection. However, dexamethasone delayed the recovery to baseline after CCI (t₁₁=2.3, P=0.04; FIG. 13, left, right), NGF (t₁₀=2.5, P=0.03; FIG. 14, left, right), and CFA (t₁₃=2.7, P=0.02; FIG. 15, left, right), such that the duration of the overall pain episode was increased by the steroid treatment; by two-fold on average after CFA. To assess whether it was the anti-inflammatory or purely analgesic actions of dexamethasone that was responsible for this prolongation, four other drugs were tested: the NSAID diclofenac, and three analgesics with no known anti-inflammatory action, systemically administered gabapentin and morphine, and peripherally administered lidocaine. All four drugs reduced allodynia during their administration period (F_{4.49}=6.1, P<0.001; FIG. 16, left, middle), but only diclofenac significantly prolonged the duration of the overall allodynia episode produced by CFA (F_{4.49}=7.5, P<0.001; FIG. 16, left, right). Diclofenac was also able to prolong the duration of CCI-induced allodynia (t₁₃=3.0, P=0.01; FIG. 25).

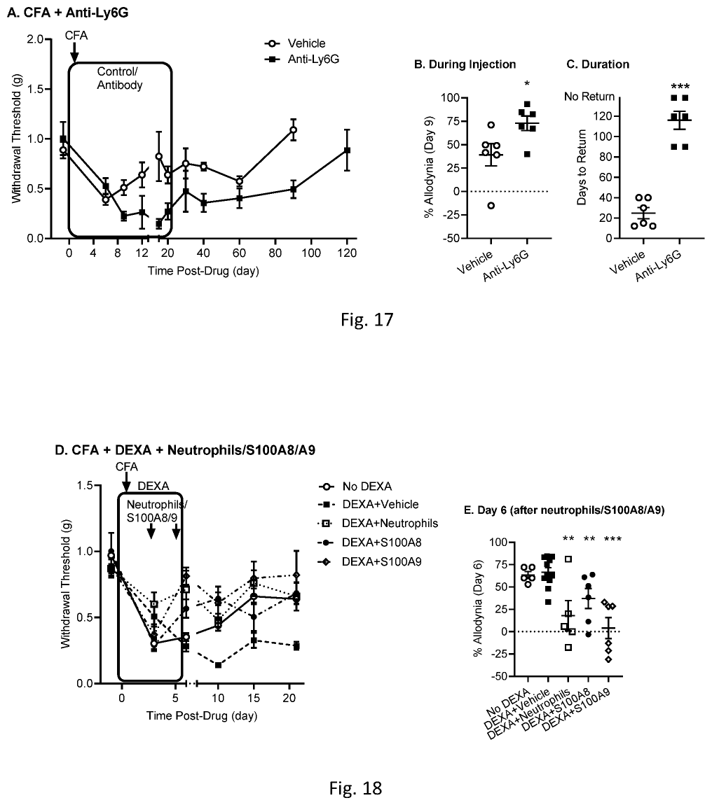

To directly assess the hypothesis that neutrophils are responsible for these effects, two complementary experiments were performed. First, neutrophils were depleted using an anti-Ly6G antibody, which causes specific but incomplete depletion (Pollenus et al., 2019). Whereas acute depletion of neutrophils using this antibody does not affect mechanical allodynia (Ghasemlou et al., 2015), prolonged administration of the antibody exacerbated allodynia (day 9: t₁₀=2.4, P=0.04; FIG. 17, left, middle) and prolonged its duration (t₁₀=8.7, P<0.001; FIG. 17, left, right) in a fashion identical to that of dexamethasone. Next, it was endeavored to determine whether the dexamethasone effect was neutrophil-dependent by injecting neutrophils isolated from peripheral blood or the neutrophil-released proteins S100A8 or S100A9 into the hind paw (ipsilateral to CFA injection) of dexamethasone-treated mice. Neutrophil injection, or injection of S100A8 or S100A9 prevented the development of allodynia entirely (comparison of DEXA-injected groups: F_{3.24}=8.7, P<0.001; FIG. 18), despite the administration of dexamethasone. This experiment also serves as a direct replication of the data shown in FIG. 15, left. In the absence of dexamethasone, neither neutrophils nor S100A8/A9 significantly affected the duration of CFA allodynia (F_{15,130}=1.1, P=0.36; FIG. 26).

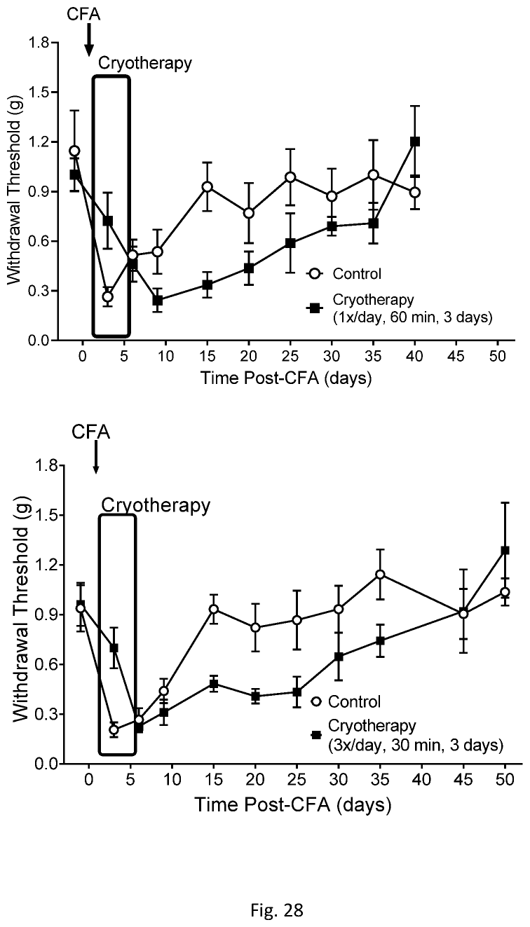

The effect of inhibiting the inflammatory response by yet another means was studied using cryotherapy in two different three-day treatment regimens (once per day for 60 min or three times per day for 30 min each). In both regimens, cryotherapy applied after CFA injection led to delayed pain recovery, despite its initial analgesic effect (FIG. 28). This further shows that inhibiting the inflammatory response (in this case with ice), can be detrimental in the long term by prolonging pain.

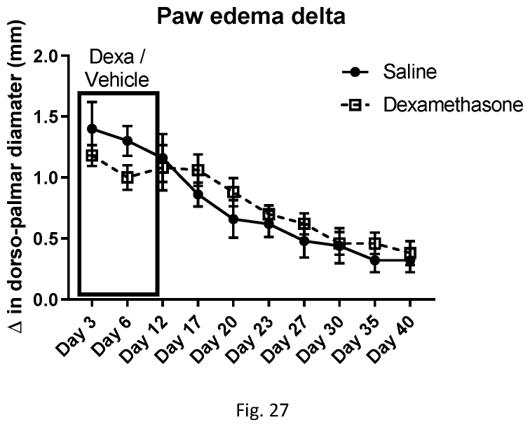

No difference in paw edema between dexamethasone or saline-treated mice were observed across 40 days post-CFA injection (FIG. 27).

All mouse experiments were performed in both sexes, and no detectable interactions with sex were observed in any experiment (all P's>0.05).

### Example 15: Prevention of the Prolongation of Postoperative Pain Caused by Dexamethasone Using a PTGIR Agonist in Mice

CD-1 mice were housed in conventional shoebox cages with 3-4 mice per cage, in temperature-controlled environment (20±1° C.), on a 12:12 h light/dark cycle, with ad libitum access to filtered water and chow (Envigo Teklad 2920x, irradiated). All mice underwent plantar incision in the right hind paw. Mice were anesthetized with 5% isoflurane at 2 L/min into a sealed induction chamber then maintained with 2.5% isoflurane at 500 mL/min through a nose cone. A 5-mm longitudinal incision was created, the flexor digitorum brevis muscle was elevated, and then a longitudinal incision was made through it. Two skin sutures closed the wound, and Polysporin antibiotic was applied. The mice were subcutaneously injected with either dexamethasone (DEXA; 0.5 mg/kg/day) or saline (PBS) once per day from the day before surgery to day 6 after surgery. Also, mice were administered 100 ug/kg/day (p.o.) of beraprost sodium, a PTGIR agonist, or saline from day 5 to 8 after plantar incision surgery. Mechanical sensitivity tests were performed using von Frey filaments (using the up-down method of Dixon) before and after the surgery on days 1, 7, 9, 14, 21 and 35. Mice were individually acclimated for at least 1 hour in transparent Plexiglas enclosures (5 cm×8.5 cm by 6 cm) before withdrawal thresholds were measured.

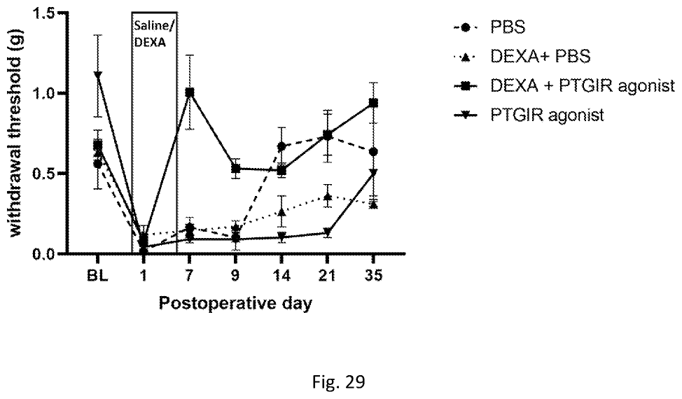

The results are shown in FIG. 29. Mice treated with PBS recovered to baseline levels after two weeks, while those treated with DEXA experienced long-lasting allodynia (i.e., pain hypersensitivity). However, the PTGIR agonist beraprost prevented long-lasting DEXA-induced hyperalgesia, and mice recovered after one week. Mice treated with only beraprost exhibited hypersensitivity for up to one month. The results showed that the PTGIR agonist prevented long-lasting allodynia following the administration of DEXA in the plantar incision surgery.

In conclusion, using a PTGIR agonist in mice successfully prevented the extension of pain caused by DEXA in a postsurgical mode. This shows the potential of prostaglandin receptor agonists as an alternative to manage chronic pain. Besides PTGIR agonists, prostaglandin receptor agonists include e.g. also PTGER4 agonists.

### Example 16: Statistical Analysis

Differential expression of genes in both LBP and TMD cohorts was assessed using moderated statistical tests implemented in the R statistical package DESeq2 (Love et al., 2014). Each test was performed with the following co-variables: sex, age, smoking status, and RIN. Pathway enrichment scores were estimated using ‘fgsea’, with statistical significance assessed using a fast permutation scheme (Sergushichev, 2016).

The meta-analysis was performed using a sample-size based analytical strategy, following the formula proposed by METAL (Willer et al., 2010). The sample sizes were: N=98 for LBP, N=30 for TMD. For each pathway and study, the sign of the enrichment score combined with its associated P-value was converted into a Z-statistic. The overall Z-statistic was obtained using a sample-size based weighted scheme. An overall P-value was calculated from the overall Z.

Pain outcomes in the LBP cohort were regressed to blood cell type fractions using the R statistical package function ‘glm’, using sex, age, smoking status as co-variables.

For transcriptomics, the false discovery rate (FDR) was relied on to correct for multiple testing, as tests are not independent of one another. Significance levels are indicated above.

For animal experiments, a criterion α=0.05 was used to determine statistical significance. Data were analyzed by Student's t-test or ANOVA followed by Tukey's or Dunnett's post-hoc testing, as appropriate.

### LIST OF REFERENCES

M. Allegri, S. Montella, F. Salici, A. Valente, M. Marchesini, C. Compagnone, M. Baciarello, M. E. Manferdini, G. Fanelli, Mechanisms of low back pain: a guide for diagnosis and therapy. F1000Res 5, (2016).
M. Ashburner, C. A. Ball, J. A. Blake, D. Botstein, H. Butler, J. M. Cherry, A. P. Davis, K. Dolinski, S. S. Dwight, J. T. Eppig, M. A. Harris, D. P. Hill, L. Issel-Tarver, A. Kasarskis, S. Lewis, J. C. Matese, J. E. Richardson, M. Ringwald, G. M. Rubin, G. Sherlock, Gene ontology: tool for the unification of biology. The Gene Ontology Consortium. Nat Genet 25, 25-29 (2000).
G. J. Bennett, Y. K. Xie, A peripheral mononeuropathy in rat that produces disorders of pain sensation like those seen in man. Pain 33, 87-107 (1988).
A. Bratus-Neuenschwander, F. Castro-Giner, M. Frank-Bertoncelj, S. Aluri, S. F. Fucentese, R. Schlapbach, H. Sprott, Pain-Associated Transcriptome Changes in Synovium of Knee Osteoarthritis Patients. Genes (Basel) 9, (2018).
F. Canonne-Hergaux, J. Calafat, E. Richer, M. Cellier, S. Grinstein, N. Borregaard, P. Gros, Expression and subcellular localization of NRAMP1 in human neutrophil granules. Blood 100, 268-275 (2002).
M. Cellier, C. Shustik, W. Dalton, E. Rich, J. Hu, D. Malo, E. Schurr, P. Gros, Expression of the human NRAMP1 gene in professional primary phagocytes: studies in blood cells and in HL-60 promyelocytic leukemia. J Leukoc Biol 61, 96-105 (1997).
S. R. Chaplan, F. W. Bach, J. W. Pogrel, J. M. Chung, T. L. Yaksh, Quantitative Assessment of Tactile Allodynia in the Rat Paw. J Neurosci Meth 53, 55-63 (1994).
C. R. Chapman, C. J. Vierck, The Transition of Acute Postoperative Pain to Chronic Pain: An Integrative Overview of Research on Mechanisms. J Pain 18, 359 e351-359 e338 (2017).
R. Chou, R. Deyo, J. Friedly, A. Skelly, M. Weimer, R. Fu, T. Dana, P. Kraegel, J. Griffin, S. Grusing, Systemic pharmacologic therapies for low back pain: a systematic review for an American College of Physicians clinical practice guideline. Ann. Intern. Med. 166, 480-492 (2017).
C. Dagostino, M. De Gregori, C. Gieger, J. Manz, I. Gudelj, G. Lauc, L. Divizia, W. Wang, M. Sim, I. K. Pemberton, J. MacDougall, F. Williams, J. Van Zundert, D. Primorac, Y. Aulchenko, L. Kapural, M. Allegri, G. PainOmics, Validation of standard operating procedures in a multicenter retrospective study to identify-omics biomarkers for chronic low back pain. PLoS One 12, e0176372 (2017).
L. Diatchenko, R. B. Fillingim, S. B. Smith, W. Maixner, The phenotypic and genetic signatures of common musculoskeletal pain conditions. Nat Rev Rheumatol 9, 340-350 (2013).
A. Dobin, C. A. Davis, F. Schlesinger, J. Drenkow, C. Zaleski, S. Jha, P. Batut, M. Chaisson, T. R. Gingeras, STAR: ultrafast universal RNA-seq aligner. Bioinformatics 29, 15-21 (2013).
S. G. Dorsey, C. L. Renn, M. Griffioen, C. B. Lassiter, S. Zhu, H. Huot-Creasy, C. McCracken, A. Mahurkar, A. C. Shetty, C. K. Jackson-Cook, H. Kim, W. A. Henderson, L. Saligan, J. Gill, L. Colloca, D. E. Lyon, A. R. Starkweather, Whole blood transcriptomic profiles can differentiate vulnerability to chronic low back pain. PLoS One 14, e0216539 (2019).
M. B. Freidin, Y. A. Tsepilov, M. Palmer, L. C. Karssen, P. Suri, Y. S. Aulchenko, F. M. K. Williams, C. M. W. Group, Insight into the genetic architecture of back pain and its risk factors from a study of 509,000 individuals. Pain 160, 1361-1373 (2019).
M. B. Freidin, Y. A. Tsepilov, I. B. Stanaway, W. Meng, C. Hayward, B. H. Smith, S. Khoury, M. Parisien, A. Bortsov, L. Diatchenko, S. Borte, B. S. Winsvold, B. M. Brumpton, J. A. Zwart, Y. S. Aulchenko, P. Suri, F. M. K. Williams, H. A.-I. Pain, Sex- and age-specific genetic analysis of chronic back pain. Pain 162, 1176-1187 (2021).
R. Freynhagen, R. Baron, U. Gockel, T. R. Tolle, painDETECT: a new screening questionnaire to identify neuropathic components in patients with back pain. Curr Med Res Opin 22, 1911-1920 (2006).
G. D. a. I. I. a. P. Collaborators, Global, regional, and national incidence, prevalence, and years lived with disability for 354 diseases and injuries for 195 countries and territories, 1990-2017: a systematic analysis for the Global Burden of Disease Study 2017. Lancet 392, 1789-1858 (2018).
The_Gene_Ontology_Consortium, The Gene Ontology Resource: 20 years and still GOing strong. Nucleic Acids Res 47, D330-D338 (2019).
N. I. George, J. F. Bowyer, N. M. Crabtree, C. W. Chang, An Iterative Leave-One-Out Approach to Outlier Detection in RNA-Seq Data. PLoS One 10, e0125224 (2015).
R. W. Gereau, 4th, K. A. Sluka, W. Maixner, S. R. Savage, T. J. Price, B. B. Murinson, M. D. Sullivan, R. B. Fillingim, A pain research agenda for the 21st century. J Pain 15, 1203-1214 (2014).
N. Ghasemlou, I. M. Chiu, J. P. Julien, C. J. Woolf, CD11b+Ly6G-myeloid cells mediate mechanical inflammatory pain hypersensitivity. Proc Natl Acad Sci USA 112, E6808-6817 (2015).
P. M. Grace, M. R. Hutchinson, S. F. Maier, L. R. Watkins, Pathological pain and the neuroimmune interface. Nat Rev Immunol 14, 217-231 (2014).
P. M. Grace, V. L. Tawfik, C. I. Svensson, M. D. Burton, M. L. Loggia, M. R. Hutchinson, The Neuroimmunology of Chronic Pain: From Rodents to Humans. J Neurosci 41, 855-865 (2021).
Y. Guo, A. M. Walsh, U. Fearon, M. D. Smith, M. D. Wechalekar, X. Yin, S. Cole, C. Orr, T. McGarry, M. Canavan, S. Kelly, T. A. Lin, X. Liu, S. M. Proudman, D. J. Veale, C. Pitzalis, S. Nagpal, CD40L-Dependent Pathway Is Active at Various Stages of Rheumatoid Arthritis Disease Progression. J Immunol 198, 4490-4501 (2017).
H. Heberle, G. V. Meirelles, F. R. da Silva, G. P. Telles, R. Minghim, InteractiVenn: a web-based tool for the analysis of sets through Venn diagrams. BMC Bioinformatics 16, 169 (2015).
M. Held, F. Karl, E. Vlckova, A. Rajdova, F. Escolano-Lozano, C. Stetter, R. Bharti, K. U. Forstner, M. Leinders, L. Dusek, F. Birklein, J. Bednarik, C. Sommer, N. Uceyler, Sensory profiles and immune-related expression patterns of patients with and without neuropathic pain after peripheral nerve lesion. Pain 160, 2316-2327 (2019).
G. Hirschfeld, B. Zernikow, Variability of “optimal” cut points for mild, moderate, and severe pain: neglected problems when comparing groups. Pain 154, 154-159 (2013).
D. Hoy, C. Bain, G. Williams, L. March, P. Brooks, F. Blyth, A. Woolf, T. Vos, R. Buchbinder, A systematic review of the global prevalence of low back pain. Arthritis Rheum 64, 2028-2037 (2012).
R. R. Ji, T. Berta, M. Nedergaard, Glia and pain: is chronic pain a gliopathy? Pain 154 Suppl 1, S10-S28 (2013).
R.-R. Ji, A. Chamessian, Y.-Q. Zhang, Pain regulation by non-neuronal cells and inflammation. Science 354, 572-577 (2016).
R. R. Ji, A. Nackley, Y. Huh, N. Terrando, W. Maixner, Neuroinflammation and Central Sensitization in Chronic and Widespread Pain. Anesthesiology 129, 343-366 (2018).
A. Kavelaars, C. J. Heijnen, Immune regulation of pain: Friend and foe. Sci Transl Med 13, eabj7152 (2021).
C. La Porta, A. Tappe-Theodor, Differential impact of psychological and psychophysical stress on low back pain in mice. Pain 161, 1442-1458 (2020).
Y. Liao, G. K. Smyth, W. Shi, featureCounts: an efficient general purpose program for assigning sequence reads to genomic features. Bioinformatics 30, 923-930 (2014).
M. I. Love, W. Huber, S. Anders, Moderated estimation of fold change and dispersion for RNA-seq data with DESeq2. Genome Biol 15, 550 (2014).
C. B. Meloto, R. Benavides, R. N. Lichtenwalter, X. Wen, N. Tugarinov, K. Zorina-Lichtenwalter, A. J. Chabot-Dore, M. H. Piltonen, S. Cattaneo, V. Verma, R. Klares, 3rd, S. Khoury, M. Parisien, L. Diatchenko, Human pain genetics database: a resource dedicated to human pain genetics research. Pain 159, 749-763 (2018).
K. A. Mifflin, B. J. Kerr, The transition from acute to chronic pain: understanding how different biological systems interact. Can J Anaesth 61, 112-122 (2014).
J. S. Mogil, J. Ritchie, S. G. Sotocinal, S. B. Smith, S. Croteau, D. J. Levitin, A. K. Naumova, Screening for pain phenotypes: analysis of three congenic mouse strains on a battery of nine nociceptive assays. Pain 126, 24-34 (2006).
A. M. Newman, C. L. Liu, M. R. Green, A. J. Gentles, W. Feng, Y. Xu, C. D. Hoang, M. Diehn, A. A. Alizadeh, Robust enumeration of cell subsets from tissue expression profiles. Nat Methods 12, 453-457 (2015).
N. A. O'Leary, M. W. Wright, J. R. Brister, S. Ciufo, D. Haddad, R. McVeigh, B. Rajput, B. Robbertse, B. Smith-White, D. Ako-Adjei, A. Astashyn, A. Badretdin, Y. Bao, O. Blinkova, V. Brover, V. Chetvernin, J. Choi, E. Cox, O. Ermolaeva, C. M. Farrell, T. Goldfarb, T. Gupta, D. Haft, E. Hatcher, W. Hlavina, V. S. Joardar, V. K. Kodali, W. Li, D. Maglott, P. Masterson, K. M. McGarvey, M. R. Murphy, K. O'Neill, S. Pujar, S. H. Rangwala, D. Rausch, L. D. Riddick, C. Schoch, A. Shkeda, S. S. Storz, H. Sun, F. Thibaud-Nissen, I. Tolstoy, R. E. Tully, A. R. Vatsan, C. Wallin, D. Webb, W. Wu, M. J. Landrum, A. Kimchi, T. Tatusova, M. DiCuccio, P. Kitts, T. D. Murphy, K. D. Pruitt, Reference sequence (RefSeq) database at NCBI: current status, taxonomic expansion, and functional annotation. Nucleic Acids Res 44, D733-745 (2016).
E. Pollenus, B. Malengier-Devlies, L. Vandermosten, T.-T. Pham, T. Mitera, H. Possemiers, L. Boon, G. Opdenakker, P. Matthys, P. E. Van den Steen, Limitations of neutrophil depletion by anti-Ly6G antibodies in two heterogenic immunological models. Immunol. Lett. 212, 30-36 (2019).
D. Ramesh, A. D'Agata, A. R. Starkweather, E. E. Young, Contribution of Endocannabinoid Gene Expression and Genotype on Low Back Pain Susceptibility and Chronicity. Clin J Pain 34, 8-14 (2018).
J. Scholz, C. J. Woolf, The neuropathic pain triad: neurons, immune cells and glia. Nat Neurosci 10, 1361-1368 (2007).
D. Schopflocher, P. Taenzer, R. Jovey, The prevalence of chronic pain in Canada. Pain Res Manag 16, 445-450 (2011).
A. W. Seifert, J. R. Monaghan, S. R. Voss, M. Maden, Skin regeneration in adult axolotls: a blueprint for scar-free healing in vertebrates. PLoS One 7, e32875 (2012).
A. Sergushichev, An algorithm for fast preranked gene set enrichment analysis using cumulative statistic calculation. bioRxiv, 10.1101/060012 (2016).
C. N. Serhan, J. Savill, Resolution of inflammation: the beginning programs the end. Nat Immunol 6, 1191-1197 (2005).
T. S. Shafshak, R. Elnemr, The Visual Analogue Scale Versus Numerical Rating Scale in Measuring Pain Severity and Predicting Disability in Low Back Pain. J Clin Rheumatol, (2020).
G. D. Slade, R. Ohrbach, J. D. Greenspan, R. B. Fillingim, E. Bair, A. E. Sanders, R. Dubner, L.
Diatchenko, C. B. Meloto, S. Smith, W. Maixner, Painful Temporomandibular Disorder: Decade of Discovery from OPPERA Studies. J Dent Res 95, 1084-1092 (2016).
G. D. Slade, J. D. Greenspan, R. B. Fillingim, W. Maixner, S. Sharma, R. Ohrbach, Overlap of Five Chronic Pain Conditions: Temporomandibular Disorders, Headache, Back Pain, Irritable Bowel Syndrome, and Fibromyalgia. J Oral Facial Pain Headache 34, s15-s28 (2020).
A. R. Starkweather, D. Ramesh, D. E. Lyon, U. Siangphoe, X. Deng, J. Sturgill, A. Heineman, R. K. Elswick, Jr., S. G. Dorsey, J. Greenspan, Acute Low Back Pain: Differential Somatosensory Function and Gene Expression Compared With Healthy No-Pain Controls. Clin J Pain 32, 933-939 (2016).
G. St Laurent, 3rd, B. Seilheimer, M. Tackett, J. Zhou, D. Shtokalo, Y. Vyatkin, M. Ri, I. Toma, D. Jones, T. A. McCaffrey, Deep Sequencing Transcriptome Analysis of Murine Wound Healing: Effects of a Multicomponent, Multitarget Natural Product Therapy-Tr14. Front Mol Biosci 4, 57 (2017).
A. Subramanian, P. Tamayo, V. K. Mootha, S. Mukherjee, B. L. Ebert, M. A. Gillette, A. Paulovich, S. L. Pomeroy, T. R. Golub, E. S. Lander, J. P. Mesirov, Gene set enrichment analysis: a knowledge-based approach for interpreting genome-wide expression profiles. Proc Natl Acad Sci USA 102, 15545-15550 (2005).
K. N. Theken, E. V. Hersh, N. F. Lahens, H. M. Lee, X. Li, E. J. Granquist, H. E. Giannakopoulos, L. M. Levin, S. A. Secreto, G. R. Grant, J. A. Detre, G. A. FitzGerald, T. Grosser, J. T. Farrar, Variability in the Analgesic Response to Ibuprofen Is Associated With Cyclooxygenase Activation in Inflammatory Pain. Clin Pharmacol Ther 106, 632-641 (2019).
J. W. S. Vlaeyen, C. G. Maher, K. Wiech, J. Van Zundert, C. B. Meloto, L. Diatchenko, M. C. Battie, M. Goossens, B. Koes, S. J. Linton, Low back pain. Nat Rev Dis Primers 4, 52 (2018).
S. Wang, R. Song, Z. Wang, Z. Jing, S. Wang, J. Ma, S100A8/A9 in Inflammation. Frontiers in Immunology 9, (2018).
C. J. Willer, Y. Li, G. R. Abecasis, METAL: fast and efficient meta-analysis of genome wide association scans. Bioinformatics 26, 2190-2191 (2010).

We claim:

1. A method of preventing or treating chronic pain in a subject in need thereof, comprising: administering to said subject one or more of:
a. a pro-inflammatory compound in an amount effective to elicit, sustain or potentiate an inflammatory process in said subject,
b. myeloid leukocyte cells in a number effective to elicit, sustain or potentiate an inflammatory process in said subject, and
c. a substance that increases the number or activity of myeloid leukocyte cells in an amount effective to elicit, sustain or potentiate an inflammatory process in said subject,
thereby preventing or treating said chronic pain.

2. The method according to claim 1, wherein said myeloid leukocyte cells are selected from the group consisting of granulocytes, monocytes and macrophages.

3. The method according to claim 2, wherein said granulocytes are neutrophils.

4. The method according to claim 1, wherein one or more of said pro-inflammatory compound, said myeloid leukocyte cells and said substance are administered during a phase of acute pain or during a phase of chronic pain.

5. The method according to claim 1, wherein said administered pro-inflammatory compound is selected from the group consisting of an alarmin, a cytokine, a growth factor, a prostaglandin receptor agonist and combinations thereof.

6. The method according to claim 5, wherein the alarmin is selected from the group consisting of S100A8 and S100A9, the cytokine is selected from the group consisting of IL-1α, IL-1β, IL-6, IL-8, TNF-α, IFN-γ, IL-12 and IL-18, the growth factor is selected from the group consisting of EREG, VEGF and GM-CSF, or the prostaglandin receptor agonist is selected from the group consisting of a PTGIR agonist and a PTGER4 agonist.

7. The method according to claim 1, wherein said administered myeloid leukocyte cells, or said myeloid leukocyte cells whose number or activity is increased, degranulate and/or release a pro-inflammatory compound.

8. The method according to claim 7, wherein the pro-inflammatory compound released by said myeloid leukocyte cells is selected from the group consisting of S100A8 and S100A9.

9. The method according to claim 1, wherein said substance that increases the number or activity of myeloid leukocyte cells is a chelator of Ca²⁺ ions.

10. The method according to claim 1, wherein said inflammatory process elicited, sustained or potentiated is transient.

11. The method according to claim 1, wherein said chronic pain is selected from the group consisting of a neuropathic pain state, a myofascial pain state, an inflammatory pain state, chronic postoperative pain, low back pain and temporomandibular disorder.

12. A method of preventing or treating chronic pain in a subject in need thereof, comprising: eliciting, sustaining, potentiating or preserving an inflammatory process in said subject, thereby preventing or treating said chronic pain.

13. The method according to claim 12, wherein eliciting, sustaining or potentiating said inflammatory process comprises administering to said subject one or more of
d. an effective amount of a pro-inflammatory compound,
e. an effective number of myeloid leukocyte cells, and
f. an effective amount of a substance that increases the number or activity of myeloid leukocyte cells.

14. A method of treating acute pain and concomitantly preventing chronic pain in a subject suffering from acute pain, comprising: classifying said subject as a subject at risk of developing chronic pain due to an insufficient inflammatory process, and administering to said subject an analgesic in an amount effective to treat said acute pain, wherein said analgesic elicits substantially no anti-inflammatory effect at the amount administered, thereby treating said acute pain and concomitantly preventing said chronic pain.

15. The method according to claim 14, wherein classifying said subject involves isolating blood from said subject, determining the amount of one or more inflammation markers in the isolated blood, and, provided that the determined amount of one, several or all inflammation markers is lower than a respective pre-determined threshold, classifying said subject as a subject at risk of developing chronic pain due to an insufficient inflammatory process.

16. The method according to claim 14, wherein said analgesic is an analgesic having substantially no anti-inflammatory properties.

17. The method according to claim 14, wherein said analgesic is selected from the group consisting of gabapentin, an opioid, lidocaine, acetaminophen (paracetamol) and an antidepressant.

18. A method of treating acute pain and concomitantly preventing chronic pain in a subject suffering from acute pain, comprising: administering to said subject a combination of (i) an analgesic in an amount effective to treat said acute pain and (ii) one or more of
g. a pro-inflammatory compound in an amount effective to elicit, sustain or potentiate an inflammatory process in said subject,
h. myeloid leukocyte cells in a number effective to elicit, sustain or potentiate an inflammatory process in said subject, and
i. a substance that increases the number or activity of myeloid leukocyte cells in an amount effective to elicit, sustain or potentiate an inflammatory process in said subject,
thereby treating said acute pain and concomitantly preventing said chronic pain.

19. The method according to claim 18, wherein said analgesic is an analgesic having or not having anti-inflammatory properties.

20. The method according to claim 18, wherein said analgesic is selected from the group consisting of an NSAID, a corticosteroid, gabapentin, an opioid, lidocaine, acetaminophen (paracetamol) and an antidepressant.

21. A method of treating acute pain and concomitantly preventing chronic pain in a subject suffering from acute pain, comprising: classifying said subject as a subject eligible for being administered an analgesic having anti-inflammatory properties and also eligible for being administered an analgesic having substantially no anti-inflammatory properties, and administering to said subject the analgesic having substantially no anti-inflammatory properties but no analgesic having anti-inflammatory properties, thereby treating said acute pain and preventing said chronic pain.

22. A method of classifying acute or chronic pain afflicting a subject, comprising:
j. isolating blood from said subject, determining the amount of one or more inflammation markers in the isolated blood, and, provided that the determined amount of one, several or all inflammation markers is lower than a respective pre-determined threshold, classifying said pain afflicting the subject as acute or chronic pain whose chronification is to be prevented or treated, respectively, and/or
k. isolating neutrophils from said subject, determining a first degree of activity of said isolated neutrophils, subsequently contacting said isolated neutrophils with a substance that increases the activity of neutrophils, determining a second degree of activity of said isolated neutrophils after being contacted with said substance, and, provided that the second degree of activity is higher than the first degree of activity and preferably that the second degree of activity is higher than a pre-determined threshold, classifying said pain afflicting the subject as acute or chronic pain whose chronification is to be prevented or treated, respectively.

23. The method according to claim 22, wherein the first and/or second degree of activity is determined by determining the amount of one or more inflammation markers.

24. The method according to claim 23, wherein the inflammation markers are selected from the group consisting of S100A8, S100A9, IL-1α, IL-1β, IL-6, TNF-α, IFN-γ, IL-12, IL-18, IL-8, EREG and GM-CSF.

25. A method of preventing or treating chronic pain in a subject suffering from acute or chronic pain, comprising: classifying the pain afflicting said subject according to the method of claim 22, and, provided that the pain afflicting said subject has been classified as acute or chronic pain whose chronification is to be prevented or treated, respectively, administering to said subject
l. a pro-inflammatory compound in an amount effective to elicit, sustain or potentiate an inflammatory process in said subject,
m. myeloid leukocyte cells in a number effective to elicit, sustain or potentiate an inflammatory process in said subject or
n. a substance that increases the number or activity of myeloid leukocyte cells in an amount effective to elicit, sustain or potentiate an inflammatory process in said subject, thereby preventing or treating said chronic pain.

26. A method of producing an animal model of chronic pain, comprising:
providing an animal,
producing a pain state in the animal,
administering to the animal, or inducing in the animal the production of, one or more of an anti-inflammatory compound and a substance that decreases the number or activity of myeloid leukocyte cells, or treating the animal so as to decrease the number or activity of myeloid leukocyte cells,
thereby producing an animal model of chronic pain.

27. The method according to claim 26, wherein producing the pain state comprises
o. administering to said animal an immunostimulant in an amount effective to produce an inflammatory pain state,
p. applying a foreign body to a nerve of said animal so as to produce a neuropathic pain state, or
q. administering to said animal a neurotrophic factor in an amount effective to produce a myofascial pain state.

28. The method according to claim 27, wherein
r. the immunostimulant is complete Freund's adjuvant,
s. applying the foreign body to said nerve of said animal comprises applying one or more ligatures around the nerve so as to produce chronic constriction injury, or
t. the neurotrophic factor is NGF.

29. The method according to claim 26, wherein said anti-inflammatory compound is selected from the group consisting of an NSAID and a corticosteroid, said substance that decreases the number or activity of myeloid leukocyte cells is an anti-Ly6G antibody, or said treating is carried out by cryotherapy.

30. An animal produced by the method of claim 26.

31. A method of modulating a pain state in an animal, comprising:
providing the animal according to claim 30, wherein the animal has a pain state, administering to the animal one or more of:
u. a pro-inflammatory compound in an amount effective to elicit, sustain or potentiate an inflammatory process in said animal,
v. myeloid leukocyte cells in a number effective to elicit, sustain or potentiate an inflammatory process in said animal, and
w. a substance that increases the number or activity of myeloid leukocyte cells in an amount effective to elicit, sustain or potentiate an inflammatory process in said animal,
thereby modulating said pain state.

32. A kit comprising a combination of (i) an analgesic and (ii) one or more of a pro-inflammatory compound, myeloid leukocyte cells and a substance that increases the number or activity of myeloid leukocyte cells.
